SEQUENCE LISTING (33 sequences)

SEQ ID NO: 1 - DNA, 852 bp, Artificial Sequence
  Synthetic
aacatccaaagacgaaaggttgaatgaaaccyytttgccatccgacatccacaggtccattctcacacaymmgtgccaaacgcaacaggaggggatacactagcagcagaccgttgcaaacgcaggacctccactcctcttctcctcaacacccacttaggctactaacaccatgactttattagcctgtctatcctggcccccctggcgttttatttccgaatgcaacaagcyccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccmsytggtcaaaaagaaacttccaaaagtcggcataccgtttgtcttgtttssyattgattgacgaatgctcaaaaataatctcattaatgcttagcgcagycyctctatcgcttctgamccccggtgcacctgtgccgaaacgcaaatggggaamcmcccgcttttyssatgattatgcattgtctccacattgtatgcttccmmgattctggtgggaatactgctgatagcctaacgttcatgatcmmaatttaacccctctaacccctactysmcagcaatatataaacagmmsgaagctgcccysycttmmmcctttyccyttatcatcattattagcttactttcataattgcgactggttccaattgacaagcttttgattttaacgacttttaactcttactagatatatcaaacttcaaagaattccgaaacg

SEQ ID NO: 2 - DNA, 852 bp, Artificial sequence
  Synthetic
aacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttaggctactaacaccatgactttattagcctgtctatcctggcccccctggcgttttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaccccccgctttttggatgattatgcattgtctccacattgtatgcttccaagattctggtgggaatactgctgatagcctaacgttcatgatcccaatttaacccctctaacccctacttgacagcaatatataaacagcccgaagctgccctgtcttccccctttccctttatcatcattattagcttactttcataattgcgactggttccaattgacaagcttttgattttaacgacttttaactcttactagatatatcaaacttcaaagaattccgaaacg

SEQ ID NO: 3 - DNA, 852 bp, Artificial sequence
  Synthetic
aacatccaaagacgaaaggttgaatgaaacctttttgccatccgacatccacaggtccattctcacacacccgtgccaaacgcaacaggaggggatacactagcagcagaccgttgcaaacgcaggacctccactcctcttctcctcaacacccacttaggctactaacaccatgactttattagcctgtctatcctggcccccctggcgttttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagcccctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaacccctctaacccctacttgacagcaatatataaacagcccgaagctgccctgtcttaaaccttttcccttatcatcattattagcttactttcataattgcgactggttccaattgacaagcttttgattttaacgacttttaactcttactagatatatcaaacttcaaagaattccgaaacg

SEQ ID NO: 4 - DNA, 852 bp, Artificial sequence
  Synthetic
aacatccaaagacgaaaggttgaatgaaacctttttgccatccgacatccacaggtccattctcacacacccgtgccaaacgcaacaggaggggatacactagcagcagaccgttgcaaacgcaggacctccactcctcttctcctcaacacccacttaggctactaacaccatgactttattagcctgtctatcctggcccccctggcgttttatttccgaatgcaacaagccccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaacccctctaacccctacttgacagcaatatataaacagaaggaagctgcccccccttaaaccttttcccttatcatcattattagcttactttcataattgcgactggttccaattgacaagcttttgattttaacgacttttaactcttactagatatatcaaacttcaaagaattccgaaacg

SEQ ID NO: 5 - DNA, 852 bp, Artificial sequence
  Synthetic
aacatccaaagacgaaaggttgaatgaaacccctttgccatccgacatccacaggtccattctcacacataagtgccaaacgcaacaggaggggatacactagcagcagaccgttgcaaacgcaggacctccactcctcttctcctcaacacccacttaggctactaacaccatgactttattagcctgtctatcctggcccccctggcgttttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccccctggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaacccctctaacccctacttgacagcaatatataaacagcccgaagctgccctgtcttccccctttccctttatcatcattattagcttactttcataattgcgactggttccaattgacaagcttttgattttaacgacttttaactcttactagatatatcaaacttcaaagaattccgaaacg

SEQ ID NO: 6 - DNA, 852 bp, Artificial sequence
  Synthetic
aacatccaaagacgaaaggttgaatgaaacccctttgccatccgacatccacaggtccattctcacacataagtgccaaacgcaacaggaggggatacactagcagcagaccgttgcaaacgcaggacctccactcctcttctcctcaacacccacttaggctactaacaccatgactttattagcctgtctatcctggcccccctggcgttttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcccaatttaacccctctaacccctacttgacagcaatatataaacagaaggaagctgcccccccttaaaccttttcccttatcatcattattagcttactttcataattgcgactggttccaattgacaagcttttgattttaacgacttttaactcttactagatatatcaaacttcaaagaattccgaaacg

SEQ ID NO: 7 - DNA, 852 bp, Artificial sequence
  Synthetic
aacatccaaagacgaaaggttgaatgaaacccctttgccatccgacatccacaggtccattctcacacataagtgccaaacgcaacaggaggggatacactagcagcagaccgttgcaaacgcaggacctccactcctcttctcctcaacacccacttaggctactaacaccatgactttattagcctgtctatcctggcccccctggcgttttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagcccctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaacccctctaacccctacttgacagcaatatataaacagcccgaagctgccctgtcttaaaccttttcccttatcatcattattagcttactttcataattgcgactggttccaattgacaagcttttgattttaacgacttttaactcttactagatatatcaaacttcaaagaattccgaaacg

SEQ ID NO: 8 - DNA, 852 bp, Artificial sequence
  Synthetic
aacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttaggctactaacaccatgactttattagcctgtctatcctggcccccctggcgttttatttccgaatgcaacaagccccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgcttttcccatgattatgcattgtctccacattgtatgcttccaagattctggtgggaatactgctgatagcctaacgttcatgatcaaaatttaacccctctaacccctacttgacagcaatatataaacagaaggaagctgcccccccttaaaccttttcccttatcatcattattagcttactttcataattgcgactggttccaattgacaagcttttgattttaacgacttttaactcttactagatatatcaaacttcaaagaattccgaaacg

SEQ ID NO: 9 - DNA, 852 bp, Artificial sequence
  Synthetic
aacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttaggctactaacaccatgactttattagcctgtctatcctggcccccctggcgttttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgacccccggtgcacctgtgccgaaacgcaaatggggaaacacccgctttttggatgattatgcattgtctccacattgtatgcttccaagattctggtgggaatactgctgatagcctaacgttcatgatcaaaatttaacccctctaacccctactccccagcaatatataaacagcccgaagctgccctgtcttaaaccttttcccttatcatcattattagcttactttcataattgcgactggttccaattgacaagcttttgattttaacgacttttaactcttactagatatatcaaacttcaaagaattccgaaacg

SEQ ID NO: 10 - DNA, 852 bp, Artificial sequence
  Synthetic
aacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttaggctactaacaccatgactttattagcctgtctatcctggcccccctggcgttttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaccccccgctttttggatgattatgcattgtctccacattgtatgcttccccgattctggtgggaatactgctgatagcctaacgttcatgatcaaaatttaacccctctaacccctacttgacagcaatatataaacagaaggaagctgcccccccttaaaccttttcccttatcatcattattagcttactttcataattgcgactggttccaattgacaagcttttgattttaacgacttttaactcttactagatatatcaaacttcaaagaattccgaaacg

SEQ ID NO: 11 - DNA, 852 bp, Artificial sequence
  Synthetic
aacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttaggctactaacaccatgactttattagcctgtctatcctggcccccctggcgttttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttcccattgattgacgaatgctcaaaaataatctcattaatgcttagcgcagtctctctatcgcttctgacccccggtgcacctgtgccgaaacgcaaatggggaaacacccgctttttggatgattatgcattgtctccacattgtatgcttccaagattctggtgggaatactgctgatagcctaacgttcatgatcaaaatttaacccctctaacccctacttgacagcaatatataaacagcccgaagctgccctgtcttccccctttccctttatcatcattattagcttactttcataattgcgactggttccaattgacaagcttttgattttaacgacttttaactcttactagatatatcaaacttcaaagaattccgaaacg

SEQ ID NO: 12 - DNA, 852 bp, Artificial sequence
  Synthetic
aacatccaaagacgaaaggttgaatgaaacctttttgccatccgacatccacaggtccattctcacacacccgtgccaaacgcaacaggaggggatacactagcagcagaccgttgcaaacgcaggacctccactcctcttctcctcaacacccacttaggctactaacaccatgactttattagcctgtctatcctggcccccctggcgttttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagcccctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaacccctctaacccctacttgacagcaatatataaacagaaggaagctgcccccccttaaaccttttcccttatcatcattattagcttactttcataattgcgactggttccaattgacaagcttttgattttaacgacttttaactcttactagatatatcaaacttcaaagaattccgaaacg

SEQ ID NO: 13 - DNA, 852 bp, Artificial sequence
  Synthetic
aacatccaaagacgaaaggttgaatgaaacctttttgccatccgacatccacaggtccattctcacacacccgtgccaaacgcaacaggaggggatacactagcagcagaccgttgcaaacgcaggacctccactcctcttctcctcaacacccacttaggctactaacaccatgactttattagcctgtctatcctggcccccctggcgttttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcccaatttaacccctctaacccctacttgacagcaatatataaacagcccgaagctgccctgtcttaaaccttttcccttatcatcattattagcttactttcataattgcgactggttccaattgacaagcttttgattttaacgacttttaactcttactagatatatcaaacttcaaagaattccgaaacg

SEQ ID NO: 14 - DNA, 852 bp, Artificial sequence
  Synthetic
aacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttaggctactaacaccatgactttattagcctgtctatcctggcccccctggcgttttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagcccctctatcgcttctgaaccccggtgcacctgtgccgaaacgcaaatggggaaacacccgctttttggatgattatgcattgtctccacattgtatgcttccccgattctggtgggaatactgctgatagcctaacgttcatgatcaaaatttaacccctctaacccctacttgacagcaatatataaacagaaggaagctgcccccccttaaaccttttcccttatcatcattattagcttactttcataattgcgactggttccaattgacaagcttttgattttaacgacttttaactcttactagatatatcaaacttcaaagaattccgaaacg

SEQ ID NO: 15 - DNA, 852 bp, Artificial sequence
  Synthetic
aacatccaaagacgaaaggttgaatgaaacccctttgccatccgacatccacaggtccattctcacacataagtgccaaacgcaacaggaggggatacactagcagcagaccgttgcaaacgcaggacctccactcctcttctcctcaacacccacttaggctactaacaccatgactttattagcctgtctatcctggcccccctggcgttttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttcccattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaacccctctaacccctacttgacagcaatatataaacagcccgaagctgccctgtcttccccctttccctttatcatcattattagcttactttcataattgcgactggttccaattgacaagcttttgattttaacgacttttaactcttactagatatatcaaacttcaaagaattccgaaacg

SEQ ID NO: 16 - DNA, 852 bp, Artificial sequence
  Synthetic
aacatccaaagacgaaaggttgaatgaaacccctttgccatccgacatccacaggtccattctcacacataagtgccaaacgcaacaggaggggatacactagcagcagaccgttgcaaacgcaggacctccactcctcttctcctcaacacccacttaggctactaacaccatgactttattagcctgtctatcctggcccccctggcgttttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaccccccgctttttggatgattatgcattgtctccacattgtatgcttccaagattctggtgggaatactgctgatagcctaacgttcatgatcaaaatttaacccctctaacccctacttgacagcaatatataaacagcccgaagctgccctgtcttccccctttccctttatcatcattattagcttactttcataattgcgactggttccaattgacaagcttttgattttaacgacttttaactcttactagatatatcaaacttcaaagaattccgaaacg

SEQ ID NO: 17 - DNA, 852 bp, Artificial sequence
  Synthetic
aacatccaaagacgaaaggttgaatgaaacccctttgccatccgacatccacaggtccattctcacacataagtgccaaacgcaacaggaggggatacactagcagcagaccgttgcaaacgcaggacctccactcctcttctcctcaacacccacttaggctactaacaccatgactttattagcctgtctatcctggcccccctggcgttttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccccgattctggtgggaatactgctgatagcctaacgttcatgatcaaaatttaacccctctaacccctacttgacagcaatatataaacagcccgaagctgccctgtcttccccctttccctttatcatcattattagcttactttcataattgcgactggttccaattgacaagcttttgattttaacgacttttaactcttactagatatatcaaacttcaaagaattccgaaacg

SEQ ID NO: 18 - DNA, 852 bp, Artificial sequence
  Synthetic
aacatccaaagacgaaaggttgaatgaaacccctttgccatccgacatccacaggtccattctcacacataagtgccaaacgcaacaggaggggatacactagcagcagaccgttgcaaacgcaggacctccactcctcttctcctcaacacccacttaggctactaacaccatgactttattagcctgtctatcctggcccccctggcgttttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccccgattctggtgggaatactgctgatagcctaacgttcatgatcaaaatttaacccctctaacccctacttgacagcaatatataaacagaaggaagctgcccccccttaaaccttttcccttatcatcattattagcttactttcataattgcgactggttccaattgacaagcttttgattttaacgacttttaactcttactagatatatcaaacttcaaagaattccgaaacg

SEQ ID NO: 19 - DNA, 852 bp, Artificial sequence
  Synthetic
aacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttaggctactaacaccatgactttattagcctgtctatcctggcccccctggcgttttatttccgaatgcaacaagccccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttcccattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaacccctctaacccctacttgacagcaatatataaacagcccgaagctgccctgtcttccccctttccctttatcatcattattagcttactttcataattgcgactggttccaattgacaagcttttgattttaacgacttttaactcttactagatatatcaaacttcaaagaattccgaaacg

SEQ ID NO: 20 - DNA, 852 bp, Artificial sequence
  Synthetic
aacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttaggctactaacaccatgactttattagcctgtctatcctggcccccctggcgttttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgacccccggtgcacctgtgccgaaacgcaaatggggaaacacccgctttttggatgattatgcattgtctccacattgtatgcttccaagattctggtgggaatactgctgatagcctaacgttcatgatcaaaatttaacccctctaacccctactccccagcaatatataaacagaaggaagctgcccccccttaaaccttttcccttatcatcattattagcttactttcataattgcgactggttccaattgacaagcttttgattttaacgacttttaactcttactagatatatcaaacttcaaagaattccgaaacg

SEQ ID NO: 21 - DNA, 852 bp, Artificial sequence
  Synthetic
aacatccaaagacgaaaggttgaatgaaacccctttgccatccgacatccacaggtccattctcacacataagtgccaaacgcaacaggaggggatacactagcagcagaccgttgcaaacgcaggacctccactcctcttctcctcaacacccacttaggctactaacaccatgactttattagcctgtctatcctggcccccctggcgttttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaacccctctaacccctactccccagcaatatataaacagaaggaagctgcccccccttaaaccttttcccttatcatcattattagcttactttcataattgcgactggttccaattgacaagcttttgattttaacgacttttaactcttactagatatatcaaacttcaaagaattccgaaacg

SEQ ID NO: 22 - DNA, 852 bp, Artificial sequence
  Synthetic
aacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttaggctactaacaccatgactttattagcctgtctatcctggcccccctggcgttttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagcccctctatcgcttctgaaccccggtgcacctgtgccgaaacgcaaatggggaaacacccgctttttggatgattatgcattgtctccacattgtatgcttccccgattctggtgggaatactgctgatagcctaacgttcatgatcaaaatttaacccctctaacccctacttgacagcaatatataaacagcccgaagctgccctgtcttaaaccttttcccttatcatcattattagcttactttcataattgcgactggttccaattgacaagcttttgattttaacgacttttaactcttactagatatatcaaacttcaaagaattccgaaacg

SEQ ID NO: 23 - DNA, 852 bp, Artificial sequence
  Synthetic
aacatccaaagacgaaaggttgaatgaaacctttttgccatccgacatccacaggtccattctcacacacccgtgccaaacgcaacaggaggggatacactagcagcagaccgttgcaaacgcaggacctccactcctcttctcctcaacacccacttaggctactaacaccatgactttattagcctgtctatcctggcccccctggcgttttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccccgattctggtgggaatactgctgatagcctaacgttcatgatcaaaatttaacccctctaacccctacttgacagcaatatataaacagcccgaagctgccctgtcttaaaccttttcccttatcatcattattagcttactttcataattgcgactggttccaattgacaagcttttgattttaacgacttttaactcttactagatatatcaaacttcaaagaattccgaaacg

SEQ ID NO: 24 - DNA, 852 bp, Artificial sequence
  Synthetic
aacatccaaagacgaaaggttgaatgaaacctttttgccatccgacatccacaggtccattctcacacacccgtgccaaacgcaacaggaggggatacactagcagcagaccgttgcaaacgcaggacctccactcctcttctcctcaacacccacttaggctactaacaccatgactttattagcctgtctatcctggcccccctggcgttttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgacccccggtgcacctgtgccgaaacgcaaatggggaaacacccgctttttggatgattatgcattgtctccacattgtatgcttccaagattctggtgggaatactgctgatagcctaacgttcatgatcaaaatttaacccctctaacccctacttgacagcaatatataaacagcccgaagctgccctgtcttaaaccttttcccttatcatcattattagcttactttcataattgcgactggttccaattgacaagcttttgattttaacgacttttaactcttactagatatatcaaacttcaaagaattccgaaacg

SEQ ID NO: 25 - DNA, 852 bp, Artificial sequence
  Synthetic
aacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttaggctactaacaccatgactttattagcctgtctatcctggcccccctggcgttttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgacccccggtgcacctgtgccgaaacgcaaatggggaaacacccgcttttcccatgattatgcattgtctccacattgtatgcttccaagattctggtgggaatactgctgatagcctaacgttcatgatcaaaatttaacccctctaacccctacttgacagcaatatataaacagcccgaagctgccctgtcttaaaccttttcccttatcatcattattagcttactttcataattgcgactggttccaattgacaagcttttgattttaacgacttttaactcttactagatatatcaaacttcaaagaattccgaaacg

SEQ ID NO: 26 - DNA, 852 bp, Artificial sequence
  Synthetic
aacatccaaagacgaaaggttgaatgaaacctttttgccatccgacatccacaggtccattctcacacacccgtgccaaacgcaacaggaggggatacactagcagcagaccgttgcaaacgcaggacctccactcctcttctcctcaacacccacttaggctactaacaccatgactttattagcctgtctatcctggcccccctggcgttttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaccccccgctttttggatgattatgcattgtctccacattgtatgcttccaagattctggtgggaatactgctgatagcctaacgttcatgatcaaaatttaacccctctaacccctacttgacagcaatatataaacagcccgaagctgccctgtcttaaaccttttcccttatcatcattattagcttactttcataattgcgactggttccaattgacaagcttttgattttaacgacttttaactcttactagatatatcaaacttcaaagaattccgaaacg

SEQ ID NO: 27 - DNA, 852 bp, Artificial sequence
  Synthetic
aacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttaggctactaacaccatgactttattagcctgtctatcctggcccccctggcgttttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttcccattgattgacgaatgctcaaaaataatctcattaatgcttagcgcagtctctctatcgcttctgacccccggtgcacctgtgccgaaacgcaaatggggaaacacccgctttttggatgattatgcattgtctccacattgtatgcttccaagattctggtgggaatactgctgatagcctaacgttcatgatcaaaatttaacccctctaacccctacttgacagcaatatataaacagcccgaagctgccctgtcttaaaccttttcccttatcatcattattagcttactttcataattgcgactggttccaattgacaagcttttgattttaacgacttttaactcttactagatatatcaaacttcaaagaattccgaaacg

SEQ ID NO: 28 - DNA, 852 bp, Artificial sequence
  Synthetic
aacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttaggctactaacaccatgactttattagcctgtctatcctggcccccctggcgttttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaccccccgctttttggatgattatgcattgtctccacattgtatgcttccccgattctggtgggaatactgctgatagcctaacgttcatgatcaaaatttaacccctctaacccctacttgacagcaatatataaacagcccgaagctgccctgtcttaaaccttttcccttatcatcattattagcttactttcataattgcgactggttccaattgacaagcttttgattttaacgacttttaactcttactagatatatcaaacttcaaagaattccgaaacg

SEQ ID NO: 29 - DNA, 852 bp, Artificial sequence
  Synthetic
aacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttaggctactaacaccatgactttattagcctgtctatcctggcccccctggcgttttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagcccctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcccaatttaacccctctaacccctacttgacagcaatatataaacagaaggaagctgcccccccttaaaccttttcccttatcatcattattagcttactttcataattgcgactggttccaattgacaagcttttgattttaacgacttttaactcttactagatatatcaaacttcaaagaattccgaaacg

SEQ ID NO: 30 - DNA, 852 bp, Artificial sequence
  Synthetic
aacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttaggctactaacaccatgactttattagcctgtctatcctggcccccctggcgttttatttccgaatgcaacaagccccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaacccctctaacccctactccccagcaatatataaacagcccgaagctgccctgtcttaaaccttttcccttatcatcattattagcttactttcataattgcgactggttccaattgacaagcttttgattttaacgacttttaactcttactagatatatcaaacttcaaagaattccgaaacg

SEQ ID NO: 31 - DNA, 852 bp, Artificial sequence
  Synthetic
aacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttaggctactaacaccatgactttattagcctgtctatcctggcccccctggcgttttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccccctggtcaaaaagaaacttccaaaagtcggcataccgtttgtcttgtttggtattgattgacgaatgctcaaaaataatctcattaatgcttagcgcagtctctctatcgcttctgacccccggtgcacctgtgccgaaacgcaaatggggaaacacccgctttttggatgattatgcattgtctccacattgtatgcttccaagattctggtgggaatactgctgatagcctaacgttcatgatcaaaatttaacccctctaacccctacttgacagcaatatataaacagcccgaagctgccctgtcttccccctttccctttatcatcattattagcttactttcataattgcgactggttccaattgacaagcttttgattttaacgacttttaactcttactagatatatcaaacttcaaagaattccgaaacg

SEQ ID NO: 32 - DNA, 852 bp, Artificial sequence
  Synthetic
aacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttaggctactaacaccatgactttattagcctgtctatcctggcccccctggcgttttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccccctggtcaaaaagaaacttccaaaagtcggcataccgtttgtcttgtttggtattgattgacgaatgctcaaaaataatctcattaatgcttagcgcagtctctctatcgcttctgacccccggtgcacctgtgccgaaacgcaaatggggaaacacccgctttttggatgattatgcattgtctccacattgtatgcttccaagattctggtgggaatactgctgatagcctaacgttcatgatcaaaatttaacccctctaacccctacttgacagcaatatataaacagcccgaagctgccctgtcttaaaccttttcccttatcatcattattagcttactttcataattgcgactggttccaattgacaagcttttgattttaacgacttttaactcttactagatatatcaaacttcaaagaattccgaaacg

SEQ ID NO: 33 - DNA, 934 bp, Pichia pastoris
aacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

(19) United States
(12) Patent Application Publication
(10) Pub. No.: US 2024/0002847 A1
(43) Pub. Date: Jan. 4, 2024

(54) SYNTHETIC METHANOL INDUCIBLE PROMOTERS AND USES THEREOF

(71) Applicant: Ginkgo Bioworks, Inc., Boston, MA (US)

(72) Inventors: Swaminath Srinivas, Melrose, MA (US); Justin Michael Gardin, Watertown, MA (US)

(73) Assignee: Ginkgo Bioworks, Inc., Boston, MA (US)

(21) Appl. No.: 18/037,215

(22) PCT Filed: Nov. 12, 2021
(86) PCT No.: PCT/US2021/059135

§ 371 Date: May 16, 2023

Related U.S. Application Data
(60) Provisional application No. 63/114,954, filed on Nov. 17, 2020.

(51) Int. Cl. C12N 15/113; C12P 21/02

(52) U.S. Cl. CPC C12N 15/113; C12P 21/02

(57) ABSTRACT

This application describes synthetic promoters capable of facilitating the high-yield synthesis of proteins and molecules.

### CROSS REFERENCE TO RELATED APPLICATIONS

This application claims priority to U.S. provisional Application No. 63/114,954, filed Nov. 17, 2020, the content of which is herein incorporated by reference in its entirety.

### SEQUENCE LISTING

In accordance with 37 C.F.R. 1.52(e)(5), the present specification makes reference to a Sequence Listing (submitted electronically as a .txt file named “G091970066WO00-SEQ”). The .txt file was generated on Nov. 5, 2021, and is 42,422 bytes in size. The Sequence Listing is herein incorporated by reference in its entirety.

### FIELD OF THE INVENTION

This disclosure relates to synthetic, methanol-inducible promoters.

### BACKGROUND

Certain methylotrophic yeast cells have been used in the production of bioproducts (e.g., proteins, nucleic acids, small molecules, etc.) due, in part, to the strong and regulatable characteristics of their native promoter systems. For example, many recombinant proteins have been successfully produced in yeast host cells in which recombinant protein production is typically driven by an endogenous methanol-regulated AOX1 promoter, P(AOX1).

It is desirable to produce a methanol-regulated promoter that is stronger than P(AOX1).

### SUMMARY

This disclosure describes synthetic promoters, host cells comprising synthetic promoters, and methods that facilitate high-yield synthesis of proteins and molecules. The synthetic promoters of the present disclosure provide advantages over P(AOX1).

Aspects of the disclosure relate to a synthetic promoter comprising a nucleic acid sequence as shown in SEQ ID NO: 1, wherein Y may be C or T, S may be G or C, and M may be A or C.

In some embodiments, wherein the synthetic promoter comprises a nucleic acid sequence as shown in SEQ ID NO: 1, the nucleotide corresponding to any one or more of positions 32, 33, 70, 71, 72, 234, 413, 414, 415, 463, 464, 465, 513, 515, 531, 567, 569, 579, 580, 581, 616, 617, 660, 661, 686, 687, 688, 706, 707, 708, 719, 720, 721, 725, 726, 727, 733 and/or 736 is a C. In some embodiments, wherein the synthetic promoter comprises a nucleic acid sequence as shown in SEQ ID NO: 1, the nucleotide corresponding to any one or more of positions 32, 33, 70, 71, 72, 234, 413, 414, 415, 463, 464, 465, 513, 515, 531, 567, 569, 579, 580, 581, 616, 617, 660, 661, 686, 687, 688, 706, 707, 708, 719, 720, 721, 725, 726, 727, 733 or 736 is a C, and all other Y bases in the sequence are T, all S bases in the sequence are G, and all M bases in the sequence are A.

Some aspects of the disclosure contemplate a synthetic promoter comprising a polynucleotide having one to thirty-eight bases different than SEQ ID NO: 33, wherein the one to thirty-eight bases that are different are located at position(s) 32, 33, 70, 71, 72, 313, 492, 493, 494, 542, 543, 544, 592, 594, 610, 646, 648, 658, 659, 660, 695, 696, 739, 740, 765, 766, 767, 785, 786, 787, 798, 799, 800, 804, 805, 806, 812, and/or 815 of a nucleic sequence as shown in SEQ ID NO: 33.

Some aspects include a synthetic promoter comprising a polynucleotide having at least 90%, at least 95%, or at least 99% identity to a nucleic sequence as shown in any one of SEQ ID NOs: 2-32.

Some aspects include a synthetic promoter comprising a polynucleotide having no more than 38 substitutions relative to a nucleic sequence as shown in any one of SEQ ID NOs: 2-32.

Some aspects contemplate a synthetic promoter having a nucleic sequence as shown in any one of SEQ ID NOs: 2-32.

Aspects of the disclosure include a transcriptional unit comprising the synthetic promoter according to any embodiment of the disclosure. In some embodiments, the synthetic promoter is operably linked to one or more genes of interest. In some embodiments, the synthetic promoter is operably linked to 1, 2, 3, 4, 5, 6, 7, 8, 9, or 10 genes of interest. In some embodiments, the synthetic promoter is operably linked to one gene of interest. In some embodiments, the synthetic promoter is operably linked to four genes of interest. In some embodiments, the synthetic promoter is operably linked to eight genes of interest.

In some embodiments, the gene of interest is expressed as an RNA. In some embodiments, the gene of interest encodes a protein. In some embodiments, the gene of interest encodes an enzyme, a structural protein, a signaling protein, a regulatory protein, a transport protein, a sensory protein, a motor protein, a defense protein, or a storage protein. In some embodiments, the protein synthesizes, modifies, or converts a molecule. In some embodiments, the gene of interest encodes Dp1B silk protein, gelatin mouse α1(I), gelatin mouse α(III), collagen human type III, cellulase, alpha-amylase, E. coli phytase, T. aquaticus subtilisin, human serum albumin, human insulin, bovine β-caesin, pertussis pertactin, tetani tetanus toxin fragment C, strep T7A endoglucanase, Aspergillus catalase L, Sc invertase, tumor necrosis factor (TNF) alpha, bovine a-lactalbumin, or bovine α-lactalbumin. In some embodiments, the protein is vaccinia capping enzyme, T7 polymerase, or O-methyltransferase.

In some embodiments, the gene of interest expresses or encodes a bioproduct. In some embodiments, a bioproduct is a nucleic acid transcribed from a gene of interest (e.g., an mRNA). In some embodiments, a bioproduct is a protein expressed from a polynucleotide (e.g., a gene of interest). In some embodiments, a bioproduct is a protein, nucleic acid (e.g., mRNA; or polynucleotide), small or large molecule, or complex or supramolecular complex (or a component of either). In some embodiments, a bioproduct is an enzyme, a structural protein, a signaling protein, a regulatory protein, a transport protein, a sensory protein, a motor protein, a defense protein, or a storage protein. In some embodiments, a bioproduct is an mRNA that encodes a viral protein. In some embodiments, a bioproduct is an mRNA that encodes a SARS-CoV-2 viral protein and is useful as a vaccine against COVID-19. In some embodiments, a SARS-CoV-2 viral protein is a spike protein. In some embodiments, a bioproduct is an mRNA that encodes a viral protein and is useful as an mRNA vaccine.

Some aspects of the invention contemplate a host cell comprising one or more synthetic promoters according to any embodiment of the disclosure and/or one or more transcriptional units according to any embodiment of the disclosure. In some embodiments, the host cell is methylotrophic. In some embodiments, the host cell is a yeast cell. In some embodiments, the host cell is from a genus of: Pichia, Komagataella, Hansenula, or Candida. In some embodiments, the host cell is Pichia pastoris, Pichia pseudopastoris, Komagataella phaffii, Pichia stipitis, Pichia membranifaciens, Komagataella pseudopastoris, Komagataella pastoris, Komagataella kurtzmanii, Komagataella mondaviorum, Hansenula polymorpha, Candida boidinii, or Pichia methanolica. In some embodiments, the host cell is Pichia pastoris. In some embodiments, one or more synthetic promoters according to any embodiment of the disclosure and/or one or more transcriptional units according to any embodiment of the disclosure are integrated into the genome of the host cell.

Some aspects include a method of engineering a host cell for protein expression comprising transforming the host cell with one or more synthetic promoters according to any embodiment of the disclosure and/or one or more transcriptional units according to any embodiment of the disclosure.

Some aspects include a method of expressing a gene of interest or producing a molecule of interest, the method comprising culturing a host cell comprising one or more synthetic promoters according to any embodiment of the disclosure and/or one or more transcriptional units according to any embodiment of the disclosure in a suitable medium. In some embodiments, the one or more synthetic promoters according to any embodiment of the disclosure and/or one or more transcriptional units according to any embodiment of the disclosure are integrated into the genome of the host cell.

Some embodiments of the methods of the disclosure include a step of extracting the expressed protein, RNA, or molecule of interest from biomass. Some embodiments of the methods of the disclosure include a step of collecting the expressed protein, RNA, or molecule of interest from culture, culture medium, cell-free spent culture medium, and/or cell-containing culture medium.

In some embodiments, the one or more synthetic promoters are methanol-inducible.

Each feature of the invention can be encompassed by various aspects of the invention. It is contemplated that each feature of the invention involving any one element or combinations of elements can be included in each embodiment of the invention. This invention is not limited in its application to the details of construction and the arrangement of components set forth in the following description or illustrated in the drawings. The invention is capable of other embodiments and of being practiced or carried out in various ways.

### BRIEF DESCRIPTION OF DRAWINGS

The accompanying drawings are not intended to be drawn to scale. The drawings are illustrative and non-limiting examples only and are not required for enablement of the disclosure. For purposes of clarity, not every component may be labeled in every drawing.

In the Drawings:

FIG. 1 depicts the test and control constructs that were integrated into the genome of yeast host cells in Example 1. The upper construct depicts a synthetic promoter of the disclosure [P(SYN)] being tested as part of a transcriptional unit expressing a gene encoding a red fluorescent protein (RFP), while the lower panel depicts P(AOX1) used in a similar transcriptional unit as a control. The genomic integrant additionally contains a transcriptional unit wherein a gene encoding a green fluorescent protein (GFP) and a hygromycin resistance (HygR) gene are linked by a 2A peptide and are expressed under a constitutive promoter P(ILV5). TT1 and TT2 are transcription terminators. This serves as an internal reference for both the test and the control constructs.

FIG. 2 shows an example of a fermentation flow diagram that depicts three stages of fermentation. Stage I, also known as the batch phase, begins with a starting culture medium containing a fixed amount of glycerol and is an initial growth phase. Stage II, the fed-batch phase, is a biomass generation phase, in which a continuous glycerol feed is maintained until sufficient biomass is accumulated. Stage III, the production phase, is marked by the transition from glycerol to methanol feeding. Additions (both as a one-time bolus or a feed) may be made throughout the fermentation process as necessary. h, hours.

FIG. 3 shows an alignment of SEQ ID NO: 33 (top; P(AOX1) promoter sequence) and SEQ ID NO: 1 (bottom; consensus sequence of SEQ ID NOs: 2-32). SEQ ID NO: 1 contains 126 gaps and 38 variable residues (shown as Y, M, or S; variable nucleotides are bolded and underlined at their respective positions in each sequence) relative to SEQ ID NO: 33.

### DETAILED DESCRIPTION

This disclosure provides synthetic promoters, host cells comprising synthetic promoters, and methods that facilitate high-yield production of desired bioproducts (e.g., without limitation, enzymes or other proteins, RNA, small molecules, etc.). “Synthetic” refers to a sequence (e.g., a nucleic acid sequence or an amino acid sequence) that is not naturally occurring. In some embodiments, a sequence that is not naturally occurring includes two or more naturally occurring sequences that are combined to form a new sequence.

In some embodiments, a synthetic promoter is operably linked to and regulates transcription of a gene of interest. The present disclosure also pertains to a host cell comprising a synthetic promoter, and to methods of using the host cell and/or synthetic promoter. In some embodiments, a host cell comprising a synthetic promoter is used to produce a bioproduct.

### Synthetic Promoters

As used in this application, a “promoter” refers to a regulatory region of DNA which directs the transcription of a sequence of DNA into RNA. In some embodiments, a promoter comprises a TATA box, or similar sequence, which is capable of directing RNA polymerase II to initiate RNA synthesis at the appropriate transcription initiation site for a particular polynucleotide sequence. In some embodiments, a promoter may additionally comprise other sequences, generally but not always positioned upstream of the TATA box, referred to as upstream promoter elements, which influence the transcription initiation rate.

In certain organisms (e.g., yeasts), a promoter, including upstream promoter elements, may be understood to encompass a sequence spanning from up to 1500 base pairs (bp) upstream of the start codon of the gene to the base abutting (e.g., immediately upstream of) the first base of the start codon of the gene. In some embodiments, the 5′-UTR region is the region of an mRNA that begins at the transcription start site and ends directly upstream from the start codon. In some embodiments, a promoter comprises a 5′-UTR, which comprises the region from the +1 position of the transcriptional start to the base abutting (immediately upstream of) the start codon (e.g., ATG) of the gene. In some embodiments, a promoter comprises the core promoter and the 5′ untranslated region (5′-UTR). For any particular promoter, the exact 5′ and 3′ ends of the promoter sequence may be defined differently by different sources, scientific references, etc. In some embodiments, the present disclosure provides synthetic promoters having a sequence as described in the appended sequence listing or shown in Table 6.

In some embodiments, the synthetic promoter comprises a polynucleotide that is at least 90%, at least 91%, at least 92%, at least 93%, at least 94%, at least 95%, at least 96%, at least 97%, at least 98%, or at least 99% identical to a nucleic acid sequence in Table 6, or to a nucleic acid sequence as shown in any one of SEQ ID NOs: 2-32, or a functional fragment thereof.

In some embodiments, the synthetic promoter comprises a polynucleotide having not more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 50 nucleotide substitutions, insertions, additions, or deletions relative to a nucleic acid sequence any one of SEQ ID NOs: 2-32, or a functional fragment thereof.

In some embodiments, the synthetic promoter comprises a polynucleotide having not more than 35 nucleotide substitutions, insertions, additions, or deletions relative to a nucleic acid sequence as shown in any one of SEQ ID NOs: 2-32, or a functional fragment thereof.

In some embodiments, the synthetic promoter has a nucleic acid sequence as shown in any one of SEQ ID NOs: 2-32, or a functional fragment thereof.

A “fragment” of a promoter refers to a portion less than the full-length promoter sequence. A “functional fragment” of a promoter of this disclosure refers to a biologically active portion of a promoter sequence. A “biologically active portion” of a genetic regulatory element, such as a promoter, comprises a portion or fragment of a full-length genetic regulatory element and has the same or similar type of activity as the full-length genetic regulatory element, although the level of activity of the biologically active portion of the genetic regulatory element may vary compared to the level of activity of the full-length genetic regulatory element.

In some embodiments, the various synthetic promoters of this disclosure share portions of nucleotide sequences with one another, such that a degree of identity (e.g., similarity) among or between synthetic promoters can be determined. In some embodiments, the degree of identity is expressed as a percentage of sequence identity. Accordingly, in some embodiments, the sequences of synthetic promoters of this disclosure are between about 97% and 99% identical to one another, including all values contained therein. In some embodiments, the degree of identity among the various synthetic promoters of the present disclosure is expressed using a consensus sequence.

A “consensus sequence” is a sequence of nucleotides which represent the most frequent residues found at each position following a sequence alignment of two or more sequences (e.g., all of the nucleic acid sequences as shown in SEQ ID NOs: 2-32). In some embodiments, where a residue is conserved among certain synthetic promoters (e.g., the 31 promoter sequences having nucleic acid sequences as shown in SEQ ID NOs: 2-32), it is shown in the consensus sequence by the single letter nucleic acid code appropriate for the conserved nucleotide (e.g., “A” for adenine, “C” for cytosine, “G” for guanine, or “T” for thymine). In some embodiments, where a nucleotide differs among or between more than one of the 31 synthetic promoters, it is shown in the consensus sequence by the single letter nucleotide code that represents the one or more differing residues that may be found at that position among the synthetic promoters. For example, where a nucleotide may be either adenine (A) or guanine (U), depending on the synthetic promoter of interest, the respective base position would be shown in a consensus sequence as “R”. This and other single letter nucleotide codes that may be used in a consensus sequence are shown in Table 1.

TABLE 1
Single-letter nucleotide codes.

| Single-letter nucleotide code | Base |
|---|---|
| A | Adenine |
| C | Cytosine |
| G | Guanine |
| T (or U) | Thymine (or Uracil) |
| R | A or G |
| Y | C or T |
| S | G or C |
| W | A or T |
| K | G or T |
| M | A or C |
| B | C or G or T |
| D | A or G or T |
| H | A or C or T |
| V | A or C or G |
| N | Any base |
| . or — | Gap, no base |

In some embodiments, the consensus sequence representing the degree of identity among the nucleic acid sequences as shown in SEQ ID NOs: 2-32 is:

(SEQ ID NO: 1)
AACATCCAAAGACGAAAGGTTGAATGAAACCYYTTTGCCATCCGACATC
CACAGGTCCATTCTCACACAYMMGTGCCAAACGCAACAGGAGGGGATAC
ACTAGCAGCAGACCGTTGCAAACGCAGGACCTCCACTCCTCTTCTCCTC
AACACCCACTTAGGCTACTAACACCATGACTTTATTAGCCTGTCTATCC
TGGCCCCCCTGGCGTTTTATTTCCGAATGCAACAAGCYCCGCATTACAC
CCGAACATCACTCCAGATGAGGGCTTTCTGAGTGTGGGGTCAAATAGTT
TCATGTTCCCCAAATGGCCCAAAACTGACAGTTTAAACGCTGTCTTGGA
ACCTAATATGACAAAAGCGTGATCTCATCCAAGATGAACTAAGTTTGGT
TCGTTGAAATGCTAACGGCCMSYTGGTCAAAAAGAAACTTCCAAAAGTC
GGCATACCGTTTGTCTTGTTTSSYATTGATTGACGAATGCTCAAAAATA
ATCTCATTAATGCTTAGCGCAGYCYCTCTATCGCTTCTGAMCCCCGGTG
CACCTGTGCCGAAACGCAAATGGGGAAMCMCCCGCTTTTYSSATGATTA
TGCATTGTCTCCACATTGTATGCTTCCMMGATTCTGGTGGGAATACTGC
TGATAGCCTAACGTTCATGATCMMAATTTAACCCCTCTAACCCCTACTY
SMCAGCAATATATAAACAGMMSGAAGCTGCCCYSYCTTMMMCCTTTYCC
YTTATCATCATTATTAGCTTACTTTCATAATTGCGACTGGTTCCAATTG
ACAAGCTTTTGATTTTAACGACTTTTAACTCTTACTAGATATATCAAAC
TTCAAAGAATTCCGAAACG.

Bolded residues represent those nucleotides which differ among two or more of the synthetic promoters of this disclosure.

In some embodiments, the synthetic promoter comprises a polynucleotide that is at least 90%, at least 91%, at least 92%, at least 93%, at least 94%, at least 95%, at least 96%, at least 97%, at least 98%, or at least 99% identical to a nucleic acid sequence as shown in SEQ ID NO: 1.

In some embodiments, wherein the synthetic promoter comprises a nucleic acid sequence as shown in SEQ ID NO: 1, the nucleotide corresponding to any one or more of positions 32, 33, 70, 71, 72, 234, 413, 414, 415, 463, 464, 465, 513, 515, 531, 567, 569, 579, 580, 581, 616, 617, 660, 661, 686, 687, 688, 706, 707, 708, 719, 720, 721, 725, 726, 727, 733 and/or 736 is a C, or (b) the nucleotide corresponding to any one or more of positions 32, 33, 70, 71, 72, 234, 413, 414, 415, 463, 464, 465, 513, 515, 531, 567, 569, 579, 580, 581, 616, 617, 660, 661, 686, 687, 688, 706, 707, 708, 719, 720, 721, 725, 726, 727, 733 or 736 is a C, and all other Y bases in the sequence are T, all S bases in the sequence are G, and all M bases in the sequence are A.

In some embodiments, a synthetic promoter is driven by (e.g., is cognate with respect to) a transcription factor and is operably linked to and capable of activating transcription of a polynucleotide encoding a gene of interest. In some embodiments, a transcription factor binds to a synthetic promoter. In some embodiments, a transcription factor necessary for transcription or increased transcription from a promoter is provided by a host cell (e.g., the genome of the host cell comprises and expresses a gene encoding the transcription factor).

Various transcription factors, and their structures and functions, are described in the literature, including: Latchman 1997 Int. J. Biochem. Cell Biology. 29 (12): 1305-12; Karin 1990 The New Biologist. 2 (2): 126-31; Babu et al. 2004 Current Opinion in Structural Biology. 14 (3): 283-91; Roeder 1996 Trends in Biochemical Sciences. 21 (9): 327-35; Nikolov et al. 1997 Proc. Nat. Acad. Sci. U.S.A. 94 (1): 15-22; Lee et al. 2000 Annual Review of Genetics. 34: 77-137; Mitchell et al. 1989 Science. 245 (4916): 371-8; Ptashne et al. 1997 Nature. 386 (6625): 569-77; Jin et al. 2014 Nucleic Acids Research. 42 (Database issue): D1182-7; and Matys et al. 2006 Nucleic Acids Research. 34 (Database issue): D108-10.

### Transcriptional Units

In some embodiments, the disclosure provides a transcriptional unit comprising a synthetic promoter. Any synthetic promoter of the present disclosure may be used in a transcriptional unit. In some embodiments, a transcriptional unit comprises a synthetic promoter and a gene of interest operably linked to the synthetic promoter. In some embodiments, the disclosure also provides a host cell comprising a transcriptional unit. In some embodiments, the disclosure provides a method comprising the step of expressing the gene of interest in a host cell comprising a transcriptional unit. In some embodiments, a gene of interest expresses a bioproduct, or contributes directly or indirectly to the production of a bioproduct (e.g., the bioproduct is synthesized, modified, or otherwise acted upon, directly or indirectly, by a protein or polynucleotide expressed from a gene of interest). In some embodiments, a gene of interest is a reporter gene (e.g., RFP or GFP) used in the construction of a synthetic promoter.

As used in this disclosure, a “transcriptional unit” refers to a sequence of nucleotides that codes for at least one RNA molecule, along with the sequences necessary for its instantiation, such as a promoter. In some embodiments, a promoter is a synthetic promoter of the disclosure. In some embodiments, a sequence of nucleotides that codes for at least one RNA molecule is a gene of interest. A “transcriptional unit” may also refer to a sequence of nucleotides that comprises a promoter (e.g., a synthetic promoter of the disclosure) operably linked to (in any order): one or more sequences of nucleotides that each code for at least one RNA molecule, and/or one or more sites suitable for insertion of a sequence of nucleotides that codes for at least one RNA molecule. A “transcriptional unit” may also refer to a sequence of nucleotides that comprises a promoter (e.g., a synthetic promoter of the disclosure) and a site suitable for insertion of a gene of interest, along with sequences necessary for its instantiation.

In some embodiments, a synthetic promoter and/or a gene of interest comprises additional sequences for expression, transcription, and/or translation of a protein encoded thereby, e.g., a 5′-UTR (5′-untranslated region), a leader sequence, and/or a 3′-UTR (3′-untranslated region), and/or one or more introns. In some embodiments, a transcriptional unit comprises one or more transcription terminators. In some embodiments, a transcriptional unit comprises one or more transcription terminators downstream of other components of the transcriptional unit.

In some embodiments, the synthetic promoter of the transcriptional unit is operably linked to one or more genes of interest. In some embodiments, a synthetic promoter is operably linked to a gene of interest that encodes an RNA. In some embodiments, a synthetic promoter is operably linked to a gene of interest that encodes a protein. In some embodiments, the gene of interest encodes an enzyme. In some embodiments, the gene of interest encodes a protein involved in the biosynthesis of an organic molecule.

A coding sequence (e.g., a gene of interest) and a regulatory sequence (e.g., a promoter sequence) are said to be “operably joined” or “operably linked” when the coding sequence and the regulatory sequence are covalently linked and/or the expression or transcription of the coding sequence is under the influence or control of the regulatory sequence. If the coding sequence is to be translated into a functional bioproduct, the coding sequence and the regulatory sequence are said to be operably joined or linked if induction of a promoter in the 5′ regulatory sequence permits the coding sequence to be transcribed and if the nature of the link between the coding sequence and the regulatory sequence does not (1) result in a frameshift event that changes the reading frame of the coding sequence, (2) interfere with the ability of the promoter region to direct the transcription of the coding sequence, or (3) interfere with the ability of the corresponding RNA transcript to be translated into a protein.

In some embodiments, the synthetic promoter is operably linked to 1, 2, 3, 4, 5, 6, 7, 8, 9, or 10 genes of interest. In some embodiments, the synthetic promoter is operably linked to one gene of interest (e.g., the transcriptional unit is monocistronic). In some embodiments, the synthetic promoter is operably linked to two or more genes of interest (e.g., the transcriptional unit is polycistronic).

In some embodiments, the disclosure provides an expression vector comprising a transcriptional unit. In some embodiments, the transcriptional unit comprises a promoter and an operably linked site suitable for insertion. In some embodiments, a gene of interest encoding a protein of interest can be inserted into the site suitable for insertion. In some embodiments, an expression vector comprising a transcriptional unit facilitates expression of a protein of interest.

In some embodiments, an insertion site is a site in a nucleic acid that is suitable for directed insertion of a polynucleotide (e.g., a synthetic or exogenous polynucleotide), including but not limited to: a gene of interest. In some embodiments, an insertion site comprises one or more restriction enzyme sites. In some embodiments, an insertion site is a multi-cloning site. In some embodiments, a multi-cloning site is a short span of a nucleic acid which comprises two or more restriction sites (e.g., EcoRI, SalI, XmaI, BamHI, SwaI, AsiSI, NotI, SacII, NheI, AccI, etc.). In some embodiments, an insertion site is a landing pad. In some embodiments, an insertion site is a landing pad, wherein the landing pad is suitable for recombinase-mediated insertion of a synthetic or exogenous polynucleotide (e.g., a synthetic promoter or a gene of interest). In some embodiments, an insertion site is a multi-landing pad site. Various landing pads and multi-landing pads are known in the art, e.g., Leonid Gaidukov et al. 2018 Nucleic Acids Res. 46(8): 4072-4086; Chi et al. 2019 PLOS ONE, Published: Jul. 25, 2019, A system for site-specific integration of transgenes in mammalian cells; and Phan et al. 2017 Nature Scientific Rep. 7:17771.

### Host Cells

In some embodiments, the present disclosure provides host cells comprising a synthetic promoter and/or a transcriptional unit comprising a synthetic promoter. Any of the synthetic promoters of the disclosure may be used in a host cell. Synthetic promoters described in this application may be introduced into a suitable host cell using any methods known in the art. In some embodiments, a host cell comprises a synthetic promoter integrated into the host cell genome.

A “host cell” refers to a cell that can be used to express a gene of interest under the control of (e.g., operably linked to) a synthetic promoter. It is understood that in some embodiments, a host cell refers not only to a particular recombinant host in which a synthetic promoter is introduced, but also to the progeny or potential progeny of such a host cell. The term “cell,” as used in this application, may refer to a single cell or a population of cells, such as a population of cells belonging to the same cell line or strain. Use of the singular term “cell” should not be construed to refer explicitly to a single cell rather than a population of cells.

Any suitable host cell may be used to express the synthetic promoters disclosed in this application, including eukaryotic cells or prokaryotic cells. Suitable host cells include, but are not limited to, fungal cells (e.g., yeast cells), bacterial cells (e.g., E. coli cells), algal cells, plant cells, insect cells, and animal cells, including mammalian cells. In some embodiments, the host cell is a yeast cell. In some embodiments, the host cell is naturally methylotrophic. A “methylotrophic cell” is one that naturally (i.e., prior to any manipulation by a human) has an ability to utilize reduced one-carbon compounds, such as methanol or methane, as the carbon source for its growth, and multi-carbon compounds that contain no carbon-carbon bonds, such as dimethyl ether and dimethylamine. Methylotrophic cells are known in the art, and include, for example, those in the genera Pichia, Komagataella, Hansenula, and Candida. A host cell that is naturally methylotrophic, such as one from among the genera Pichia, Komagataella, Hansenula, or Candida but has been rendered unable to utilize methanol, e.g. by engineering, is still considered to be a methylotrophic host cell for purposes of this disclosure. In some embodiments, the host cell is not naturally methylotrophic.

In some embodiments, a host cell includes any of: a member of the genera Pichia, Komagataella, Candida, Dipodascus, Galactomyces, Hansenula, Kluyveromyces (e.g., K. lactis), Magnusiomyces, Ogatae, Phaffomyces, Saccharomyces (e.g., S. cerevisiae), Schizosaccharomyces, Starmera, Starmerella, Sugiyamaella, Trichomonascus, Wickerhamomyces, Wickerhamiella, Williopsis, Yarrowia, or Zygoascus; or a member of Komagataella Clade, Phaffomyces Clade, Dipodascaceae, Phaffomycetaceae, or Trichomonascaceae. In some embodiments, the host cell is a member of the genera Pichia or Komagataella. In some embodiments, the host cell is a Pichia pastoris, Pichia pseudopastoris, Komagataella phaffii, Pichia stipitis, Pichia membranifaciens, Komagataella pastoris, Komagataella pseudopastoris, Komagataella kurtzmanii, Komagataella mondaviorum, Hansenula polymorpha, Candida boidinii, or Pichia methanolica cell. In some embodiments, the host cell is any of a: Pichia pastoris, Pichia pseudopastoris, Pichia stipitis, Pichia membranifaciens, Pichia methanolica, Pichia finlandica, Pichia trehalophila, Pichia kodamae, Pichia opuntiae, Pichia therrnotolerans, Pichia salictaria, Pichia quercuum, Pichia pijperi, Pichia angusta, Komagataella phaffii, Komagataella pastoris, Komagataella pseudopastoris, Komagataella kurtzmanii, Komagataella mondaviorum, Wickerhamomyces anomalus, Candida albicans, Candida lusitaniae, Ogataea glucozyma, Candida blankii, Candida boidinii, Candida orba, Candida petrohuensis, Candida santjacobensis, Candida sorboxylosa, Candida sp., Dipodascus albidus, Galactomyces geotrichum, Hansenula polymorpha, Kluyveromyces lactis, Magnusiomyces magnusii, Phaffomyces antillensis, Phaffomyces opuntiae, Phaffomyces thermotolerans, Saccharomyces cerevisiae, Saccharomyces carlsbergensis, Saccharomyces diastaticus, Saccharomyces norbensis, Saccharomyces kluyveri, Schizosaccharomyces pombe, Starmerella bombicola, Sugiyamaella smithiae, Trichomonascus petasosporus, Wickerhamiella domercqiae, Yarrowia lipolytica, or Zygoascus hellenicus cell. In some embodiments, a host cell is an undescribed species of Pichia or Komagataella. In some embodiments, a host cell is a Pichia sp. or Komagataella sp.

In some embodiments, the yeast strain is an industrial yeast strain. In some embodiments, the host cell is a fungal cell. In some embodiments, a fungal cell includes a cell of Aspergillus spp., Penicillium spp., Fusarium spp., Rhizopus spp., Acremonium spp., Neurospora spp., Sordaria spp., Magnaporthe spp., Allomyces spp., Ustilago spp., Botrytis spp., or Trichoderma spp.

Without wishing to be bound by any particular theory, the present disclosure notes that some reports in the scientific literature reassigned P. pastoris to the genus Komagataella, and various strains of P. pastoris were separated into K. phaffii, K. pastoris, and K. pseudopastoris. In some embodiments, Pichia pastoris is identical to Komagataella phaffii, and Komagataella phaffii is sometimes referred to by its former species name Pichia pastoris. As used in this disclosure, Pichia pseudopastoris is interchangeable with Komagataella pseudopastoris. These various genera and species, and the relationships between them, are described in the scientific literature, for example: Feng et al. 2020 Yeast 37(2):237-245; De Schutter et al. 2009. Nature Biotechnology. 27 (6): 561-566; Heistinger et al. 2018 Molecular and Cellular Biology 38 Issue 2 e00398-17; Kurtzman, International Journal of Systematic and Evolutionary Microbiology (2005), 55: 973-976; Kurtzman 2011 Antonie van Leeuwenhoek 99:13-23; Kurtzman 2013 Antonie van Leeuwenhoek 104:339-347; Kurtzman 2012 Antonie van Leeuwenhoek 101: 859-868; Naumov 2018 Antonie van Leeuwenhoek 111:1197-1207; and Yamada et al. 1995 Biosci. Biotech. Biochem. 59: 439-444.

In some embodiments, the host cell is an algal cell such as Chlamydomonas (e.g., C. reinhardtii) and Phormidium (P. sp. ATCC29409).

In some embodiments, the host cell is a prokaryotic cell. Suitable prokaryotic cells include gram positive, gram negative, and gram-variable bacterial cells.

Various strains that may be used as host cells in the practice of the disclosure are readily accessible to the public from a number of culture collections such as American Type Culture Collection (ATCC), Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL).

A host cell may comprise genetic modifications relative to a wild-type counterpart, in addition to harboring the synthetic promoter. In some embodiments, a host cell is modified to reduce or inactivate one or more endogenous genes. Reduction of gene expression and/or gene inactivation may be achieved through any suitable method, including but not limited to deletion of the gene, introduction of a point mutation into the gene, truncation of the gene, introduction of an insertion into the gene, introduction of a tag or fusion into the gene, or selective editing of the gene. For example, polymerase chain reaction (PCR)-based methods may be used (see, e.g., Gardner et al., Methods Mol Biol. 2014; 1205:45-78) or gene-editing techniques may be used. As a non-limiting example, genes may be deleted through gene replacement (e.g., with a marker, including a selection marker). A gene may also be truncated through the use of a transposon system (see, e.g., Poussu et al., Nucleic Acids Res. 2005; 33(12): e104).

In some embodiments, a host cell expresses an RNA polymerase, a transcription factor(s), and any other cellular components necessary for transcription from a synthetic promoter. In some embodiments, a host cell expresses an RNA polymerase, a transcription factor(s), and any other cellular components necessary for transcription from P(AOX1). In some embodiments, P(AOX1) is a control promoter.

Some aspects of the present disclosure describe a method of engineering a host cell for protein expression comprising transforming the host cell with one or more synthetic promoters and/or one or more transcriptional units of the present disclosure. In some embodiments, one or more synthetic promoters and/or one or more transcriptional units of the present disclosure are integrated into the genome of the host cell. Any synthetic promoter or transcriptional unit of the present disclosure may be used.

### Culturing of Host Cells

Any of the host cells comprising one or more synthetic promoters and/or transcriptional units comprising a synthetic promoter(s) may be cultured under any suitable conditions, including, but not limited to, the culture conditions described in this disclosure and/or known in the art, and may use any method and be conducted in media of any type (e.g., rich and/or minimal and/or nutrient-limiting, etc.). For example, any media, temperature, and incubation conditions known in the art may be used. Example culture conditions are provided in this disclosure. For host cells comprising an inducible promoter, cells may be cultured with an appropriate agent (e.g., methanol) to induce expression. In some embodiments, the culture conditions may be used to control the timing and/or level of expression of a gene of interest operably linked to a synthetic promoter and/or production of a bioproduct.

In some embodiments, culturing of host cells comprising a synthetic promoter occurs over several phases or stages. The terms “stage” and “phase” are used interchangeably in this application. In some embodiments, it may be desirable to limit expression of a gene of interest until a later phase, e.g., the production phase, as expression or high expression of the gene of interest may cause toxicity and/or otherwise reduce cell growth. Without wishing to be bound by any particular theory, the present disclosure notes that, even in a relatively tightly controlled genetic system, a low or basal level of expression of a gene of interest may occur prior to production phase, but if such expression leads to toxicity and/or decreases growth rate(s), the cells can be maintained under conditions to decrease the expression to as low a level as technically feasible.

As a non-limiting example, the culturing conditions of a host cell comprising a synthetic promoter or transcriptional unit comprising a synthetic promoter of this disclosure can be altered in production phase, such that the synthetic promoter is induced and a high level of expression of the gene of interest is achieved.

In some embodiments, host cells comprise one or more transcriptional units comprising a synthetic promoter(s) operably linked to gene(s) of interest, and culturing of host cells occurs over the stages of: Stage I, Stage II, and Stage III. In some embodiments, in Stage I (also known as the batch phase), fresh, sterile medium is initially inoculated with host cells. After a period of growth, the culture from Stage I is ready for the subsequent phase. In some embodiments, in Stage II (also known as a fed-batch or cell growth phase), the cultures grow, and biomass increases. In some embodiments, in at least part of Stage II, cell growth is exponential. In some embodiments, in Stage III (also known as a production phase or induction phase), the synthetic promoter, if not already induced, is induced (e.g., by the addition of exogenously supplied methanol) to express the gene of interest. In some embodiments, the promoter is not induced in Stage I or Stage II, but is induced during Stage III, allowing high expression of the gene of interest. In some embodiments, during a production phase, an additional component is added to the culture medium. In some embodiments, the additional component is a nutrient. In some embodiments, the additional component further increases expression from the synthetic promoter. In some embodiments, the additional component is methanol.

In some embodiments, the culturing process includes a batch phase, in which the nutrient is maintained at excess, and a fed-batch phase, wherein the culture is step-fed to maintain excess levels of the nutrient. In some embodiments, the batch phase can be considered the last part of Stage I, and is followed by the fed-batch phase in Stage II.

The various stages can also occur using the same or different growth media, volumes, duration, temperatures (e.g., 30° C., 35° C., 37° C., or 42° C.), pH levels (e.g., acidic, slightly acidic, neutral, slightly basic, or basic), agitation levels, aeration levels, dissolved oxygen levels, levels and/or concentrations and/or flowrates of the limiting nutrient, additional nutrients, conditions, etc. As is known in the art, and as appropriate for differences in culture volumes and cell density, the various stages can occur in any vessel and do not need to occur in the same type or size of vessel.

In some embodiments, host cells can be cultured in an industrial-scale process. In some embodiments, industrial-scale processes are operated in continuous, semi-continuous or non-continuous modes. Non-limiting examples of operation modes are batch, fed batch, extended batch, repetitive batch, draw/fill, rotating-wall, spinning flask, and/or perfusion modes of operation. In some embodiments, a bioreactor, fermentor, or other vessel includes a sensor and/or a control system to measure and/or adjust reaction parameters. Non-limiting examples of reaction parameters include biological parameters (e.g., growth rate, cell size, cell number, cell density, cell type, or cell state, etc.), chemical parameters (e.g., pH, redox-potential, concentration of reaction substrate and/or product, concentration of dissolved gases, nutrient concentrations, metabolite concentrations, etc.), and physical/mechanical parameters (e.g., density, conductivity, degree of agitation, pressure, and flow rate, etc.).

The culture medium may comprise various components, including, but not limited to: potassium, potassium phosphate monobasic, ammonium, ammonium sulfate, calcium, calcium sulfate dihydrate, potassium sulfate, magnesium, magnesium sulfate heptahydrate, a trace metal, PTM4 solution, copper, copper (II) sulfate pentahydrate, sodium iodide, manganese, manganese (II) sulfate monohydrate, sodium, molybdenum, sodium molybdate dihydrate, boric acid, cobalt, cobalt (II) chloride (anhydrous), zinc, zinc chloride (anhydrous), iron, iron (II) sulfate heptahydrate, biotin, sulfate, sulfuric acid, water, and/or other optional nutrients (which can be present, present in abundance, present in excess, or limiting; e.g., the nutrient is absent or not exogenously added to the medium). The medium can be sterilized by any method known in the art.

In some embodiments, the culture medium comprises a carbon source. In some embodiments, a carbon source(s) during fermentation (e.g., a growth phase such as Stage I and/or Stage II) is: glucose; glycerol and/or sorbitol; or glycerol and/or sorbitol. In some embodiments, a carbon source during fermentation (e.g., a growth phase such as Stage I and/or Stage II) is glycerol. In some embodiments, a carbon source(s) during production (e.g., a production phase such as Stage III) is: methanol; or methanol and glycerol. In some embodiments, a carbon source during production (e.g., a production phase such as Stage III) is methanol. In some embodiments, the carbon sources during production (e.g., a production phase such as Stage III) are methanol and glycerol.

Example 3 shows various culture conditions useful for culturing host cells of the present disclosure. A variety of culture media suitable for various vessels, purposes, and host cells are described in this document (e.g., in Example 3 and throughout the disclosure) and/or are generally known in the art.

### Expression of Genes of Interest in Host Cells

Aspects of the present disclosure contemplate a method of expressing a gene of interest or producing a molecule of interest, the method comprising culturing a host cell comprising one or more transcriptional units comprising a synthetic promoter(s) operably linked to a gene(s) of interest, in a suitable medium to allow for cell growth. In some embodiments, the one or more synthetic promoters and/or one or more transcriptional units are integrated into the genome of the host cell. Any synthetic promoter or transcriptional unit of the present disclosure may be used. The host cell may be any host cell of the present disclosure.

In some embodiments, the expressed genes of interest are synthetic. In some embodiments, a synthetic gene of interest that is introduced into the host cell may be a polynucleotide that comes from a different organism, genus, or species from the host cell; or a synthetic, engineered, or chimeric polynucleotide, or a polynucleotide that is also endogenously expressed in the same organism or species as the host cell but has been altered. For example, a polynucleotide that is endogenously present in a host cell may be considered synthetic when it is altered to be: situated non-naturally in the host cell; expressed recombinantly in the host cell, either stably or transiently; modified within the host cell; selectively edited within the host cell; expressed in a copy number that differs from the naturally occurring copy number within the host cell; or expressed in a non-natural way within the host cell, such as by manipulating regulatory regions that control expression of the polynucleotide.

In some embodiments, a gene of interest is a polynucleotide that is endogenously present in a host cell and whose expression is driven by a synthetic promoter that does not naturally regulate expression of the polynucleotide. In some embodiments, the synthetic promoter is activated or repressed by a recombinant molecule. For example, gene editing-based techniques may be used to regulate expression of a polynucleotide, including an endogenous polynucleotide, from a synthetic promoter. See, e.g., Chavez et al., Nat Methods. 2016 Jul; 13(7): 563-567. A gene of interest may comprise a variant sequence as compared with a reference polynucleotide sequence; or may comprise a wild-type sequence but may not be in the wild-type context within a genome (e.g., a wild-type sequence that is expressed in/by a host cell or in a chromosomal location where it is not normally expressed).

In some embodiments, the gene of interest encodes an enzyme, a structural protein, a signaling protein, a regulatory protein, a transport protein, a sensory protein, a motor protein, a defense protein, or a storage protein. In some embodiments, the gene of interest encodes a vaccinia capping enzyme, a T7 polymerase enzyme, or an O-methyltransferase enzyme. In some embodiments, the gene of interest encodes Dp1B Silk protein, gelatin mouse al(I), gelatin mouse α(III), collagen human Type III, cellulase, alpha-amylase, E. coli phytase, T. aquaticus subtilisin, human serum albumin, human insulin, bovine β-caesin, pertussis pertactin, tetani tetanus toxin fragment C, strep T7A endoglucanase, Aspergillus catalase L, Sc invertase, tumor necrosis factor (TNF) alpha, bovine a-lactalbumin, or bovine a-lactalbumin.

In some embodiments, the coding sequence of the gene of interest may be codon optimized for expression in a particular host cell, including, but not limited to, a Pichia pastoris, Pichia pseudopastoris, Komagataella phaffii, Pichia stipitis, Pichia membranifaciens, Komagataella pastoris, Komagataella pseudopastoris, Komagataella kurtzmanii, Komagataella mondaviorum, Hansenula polymorpha, Candida boidinii, or Pichia methanolica cell.

Bioproducts Expressed from Genes of Interest

In some embodiments, the present disclosure pertains to a host cell comprising a synthetic promoter, wherein, when the host cell is cultured, the host cell is capable of producing a bioproduct (e.g., a molecule of interest).

In some embodiments, a bioproduct is a protein expressed from a polynucleotide (e.g., a gene of interest). In some embodiments, a bioproduct is any composition that is synthesized, modified, or otherwise acted upon, directly or indirectly, by a protein or polynucleotide expressed from a gene of interest.

The synthetic promoters, host cells, and other methods described in this disclosure can therefore be used for and/or facilitate the high-yield, large-scale production of bioproducts. In some embodiments, the bioproduct is obtained from biomass or culture. In some embodiments, obtaining the bioproduct comprises extracting the bioproduct from biomass. In some embodiments, obtaining the bioproduct comprises collecting the bioproduct from the culture medium.

In some embodiments, methods of producing a bioproduct are provided, comprising the steps of: expressing a gene of interest by culturing a host cell, purifying an enzyme encoded by the gene of interest, and using the purified enzyme for bioconversion of a substrate to a molecule of interest.

The term “bioproduct” refers to any product that is made by or from biomass. “Biomass” refers to any biological material that is available on a renewable basis, including by production in any host cells.

In some embodiments, a bioproduct is a protein, nucleic acid (e.g., mRNA; or polynucleotide), small or large molecule, or complex or supramolecular complex (or a component of either). In some embodiments, a bioproduct is an enzyme, a structural protein, a signaling protein, a regulatory protein, a transport protein, a sensory protein, a motor protein, a defense protein, or a storage protein. In some embodiments, a bioproduct is a compound or composition that is synthesized (in whole or in part), modified, and/or converted, directly or indirectly, into another, a final, or a more useful or stable form by the action of the protein or nucleic acid encoded by a gene of interest. In some embodiments, the gene of interest is expressed as an RNA.

In some embodiments, the gene of interest encodes a protein. In some embodiments, the protein is an enzyme, a structural protein, a signaling protein, a regulatory protein, a transport protein, a sensory protein, a motor protein, a defense protein, or a storage protein.

In some embodiments, the protein is an enzyme. In some embodiments, the enzyme (e.g., protein) is vaccinia capping enzyme, T7 polymerase, or O-methyltransferase.

In some embodiments, the protein synthesizes, modifies, or converts a molecule.

In some embodiments, one or more synthetic promoters are used to produce a protein of interest in a host cell.

In some embodiments, a bioproduct is a nucleic acid transcribed from a gene of interest (e.g., an mRNA). In some embodiments, a bioproduct is an mRNA that encodes a viral protein. In some embodiments, a bioproduct is an mRNA that encodes a SARS-CoV-2 viral protein and is useful as a vaccine against COVID-19. In some embodiments, a SARS-CoV-2 viral protein is a spike protein. In some embodiments, a bioproduct is an mRNA that encodes a viral protein and is useful as an mRNA vaccine. In some embodiments, the bioproduct is a vaccinia capping enzyme. In some embodiments, the bioproduct is an O-methyltransferase or T7 polymerase.

In some embodiments, a bioproduct is (e.g., the gene of interest encodes) Dp1B silk protein, gelatin mouse al(I), gelatin mouse α(III), collagen human Type III, cellulase, alpha-amylase, E. coli phytase, T. aquaticus subtilisin, human serum albumin, human insulin, bovine β-caesin, pertussis pertactin, tetani tetanus toxin fragment C, strep T7A endoglucanase, Aspergillus catalase L, Sc invertase, tumor necrosis factor (TNF) alpha, bovine a-lactalbumin, or bovine a-lactalbumin.

In some embodiments, a bioproduct is (e.g., the gene of interest encodes) myoglobin, beta-lactoglobulin, ovalbumin, alpha-lactalbumin, caseins (alpha S1, S2, beta, kappa), lactoferrin, transglutaminase, or osteopontin.

In some embodiments, the bioproduct is a small molecule.

In some embodiments, a bioproduct is a small or large molecule which is synthesized (in whole or in part), modified, and/or converted into another, a final, or a more useful or stable form, directly or indirectly, by the action of a protein expressed from a gene of interest.

In some embodiments, a bioproduct is a component (e.g., a protein, nucleic acid, small or large molecule, etc.) which is useful in a bioconversion process.

### Measuring Bioproducts

The amount of production of a bioproduct may be evaluated at any one or multiple steps of a pathway, such as a final product or an intermediate product, using metrics familiar to those of skill in the art. Production may be assessed by any metric known in the art, for example, by assessing volumetric productivity, enzyme kinetics/reaction rate, specific productivity, biomass-specific productivity, titer, yield, and total titer of one or more bioproducts.

In some embodiments, the metric used to measure production may depend on whether a continuous process is being monitored or whether a particular end product is being measured. For example, in some embodiments, metrics used to monitor production by a continuous process may include volumetric productivity, enzyme kinetics, and reaction rate. In some embodiments, metrics used to monitor production of a particular product may include specific productivity, biomass-specific productivity, activity, titer, and/or yield of one or more bioproducts. The term “volumetric productivity” or “production rate” refers to the amount of product formed per volume of medium per unit of time. Volumetric productivity can be reported in grams per liter per hour (g/L/h).

It should be appreciated that bioproducts can be measured by any means known to one of ordinary skill in the art. In some embodiments, bioproduct production may be determined by measuring the amount of bioproduct produced per unit biomass per unit time. For example, the bioproducts may be measured in, e.g., mmol bioproduct produced per liter of fermentation medium per hour. In some embodiments, a host cell comprising a synthetic promoter of this disclosure may produce at least 0.1 mmol (e.g., at least 1 mmol, at least 1.5 mmol, at least 2 mmol, at least 2.5 mmol, at least 3, at least 3.5 mmol, at least 4 mmol, at least 4.5 mmol, at least 5 mmol, or at least 10 mmol of bioproduct, including all values in between).

In some embodiments, the level of bioproducts may be determined by, e.g., comparing the quantity or amount of bioproduct produced by a host cell comprising a synthetic promoter of this disclosure to a control host cell. In some embodiments, the host cell comprising a synthetic promoter of this disclosure provides for production of a bioproduct encoded by the gene of interest at a level that that is higher than the level of the bioproduct produced in a control host cell. In some embodiments, the control host cell is a cell that comprises a methanol-inducible promoter, such as P(AOX1) of P. pastoris, operably linked to a gene of interest. In some embodiments, the gene of interest encoded by the control host cell is the same gene of interest encoded by the host cell comprising a synthetic promoter of this disclosure. In some embodiments, a gene of interest is a reporter gene. In some embodiments, the control host cell and the host cell comprising a synthetic promoter of this disclosure are of the same species. In some embodiments, the control host cell comprises an endogenous promoter and is cultured in the same or different conditions as or from a host cell that comprises the synthetic promoter, wherein the host cells are of the same type.

In some embodiments, a control host cell is a wild-type cell, such as a wild-type Pichia pastoris, Komagataella phaffii, Komagataella pastoris, Komagataella pseudopastoris, Hansenula polymorpha, Candida boidinii, or Pichia methanolica cell. In some embodiments, the control host cell comprises a synthetic promoter that is identical to a synthetic promoter expressed in a host cell of a different type.

In some embodiments, the concentration (or quantity, amount, etc.) of bioproduct produced by a host cell comprising a synthetic promoter of this disclosure is at least 1.1 fold (e.g., at least 1.3 fold, at least 1.5 fold, at least 1.7 fold, at least 1.9 fold, at least 2 fold, at least 2.5 fold, at least 3 fold, at least 4 fold, at least 5 fold, at least 10 fold, at least 20 fold, at least 30 fold, at least 40 fold, at least 50 fold, or at least 100 fold, including all values in between) greater than that of a control host cell or the same host cell that does not comprise the synthetic promoter.

In some embodiments, a host cell that comprises a synthetic promoter of this disclosure produces at least 10%, 20%, 30%, 40%, 50%, 60%, 70%, 80%, 90%, or 100% more bioproduct compared to a control host cell or the same host cell that does not comprise the synthetic promoter.

In some embodiments, a host cell comprising a synthetic promoter of this disclosure is capable of producing at least 5 g/L, 10 g/L, at least 15 g/L, at least 20 g/L, at least at least 25 g/L, at least 30 g/L, at least 35 g/L, or at least 40 g/L of one or more bioproducts.

In some embodiments, the potency of a synthetic promoter is evaluated based on the amount of bioproduct generated in specific culture phases (e.g., growth phase, production phase, etc.). Excess bioproduct generated in the growth phase may be an indication of non-specific, or “leaky,” promoter activity, which may be undesirable. In some embodiments, the amount of bioproduct produced using a synthetic promoter of the present disclosure is greater in the production phase than in the growth phase. In some embodiments, the amount of bioproduct produced using a synthetic promoter of the present disclosure in the production phase is greater than that which can be produced in the production phase by a control cell or the same host cell that does not comprise the synthetic promoter. In some embodiments, the amount of bioproduct produced using a synthetic promoter of the present disclosure in the production phase is 1%, 2%, 3%, 5%, 10%, 20%, 30%, 40%, 50%, 60%, 70%, 80%, 90%, 100%, 150%, 200%, or any value greater than that which can be produced in the production phase by a control host cell or the same host cell that does not comprise the synthetic promoter.

In some embodiments, the amount of bioproduct produced using a synthetic promoter of the present disclosure is less in the growth phase than in the production phase. In some embodiments, the amount of bioproduct produced using a synthetic promoter of the present disclosure in the growth phase is less than that which is produced in the growth phase by a control host cell or the same host cell that does not comprise the synthetic promoter. In some embodiments, the amount of bioproduct produced using a synthetic promoter of the present disclosure in the growth phase is 1%, 2%, 3%, 5%, 10%, 20%, 30%, 40%, 50%, 60%, 70%, 80%, 90%, or 100% less than that which is produced in the growth phase by a control host cell or the same host cell that does not comprise the synthetic promoter.

In some embodiments, the efficiency of a synthetic promoter may be expressed as a ratio of bioproduct expressed in the growth phase versus the bioproduct expressed in the production phase (e.g., 1:1, 1:2, 1:3, etc.). In some embodiments, the ratio of bioproduct expressed in the growth phase versus the bioproduct expressed in the production phase using a synthetic promoter of the present disclosure is about 1:1.1, about 1:1.2, about 1:1.3, about 1:1.4, about 1:1.5, about 1:1.6, about 1:1.7, about 1:1.8, about 1:1.9, about 1:2, about 1:3, about 1:4, about 1:5, about 1:6, about 1:7, about 1:8, about 1:9, about 1:10, about 1:20, about 1:30, about 1:40, about 1:50, about 1:60, about 1:70, about 1:80, about 1:90, about 1:100, about 1:150, about 1:200, or any ratio included therein.

In some embodiments, any of the methods described in this application may include isolation and/or purification of products of the expression of genes of interest (e.g., proteins and/or nucleic acids). For example, the isolation and/or purification can involve one or more of cell lysis, centrifugation, extraction, column chromatography, distillation, crystallization, and/or lyophilization.

Products produced by any of the host cells expressing the synthetic promoters disclosed in this application, or any of the in vitro methods described in this application, may be identified, isolated, extracted, and/or purified using any method known in the art. Mass spectrometry (e.g., LC-MS, GC-MS) is a non-limiting example of a method for identification and may be used to analyze the chemical composition and/or chemical structure and/or concentration of a compound of interest.

### Variants

Aspects of the disclosure relate to polynucleotides, including polynucleotides encoding synthetic promoters. Variants of the polynucleotides described in this application are also encompassed by this disclosure. A variant may share at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with a reference sequence, including all values in between. In some embodiments, the disclosure provides variants of a synthetic promoter.

Unless otherwise noted, the term “sequence identity,” as known in the art, refers to a relationship between the sequences of two polynucleotides, as determined by sequence comparison (alignment). In some embodiments, sequence identity is determined across the entire length of a sequence, while in other embodiments, sequence identity is determined over a region of a sequence. “Identity” can also refer to the degree of sequence relatedness between two sequences as determined by the number of matches between strings of two or more residues (e.g., nucleic acid residues). Identity measures the percent of identical matches between the smaller of two or more sequences with gap alignments (if any) addressed by a particular mathematical model, algorithms, or computer program.

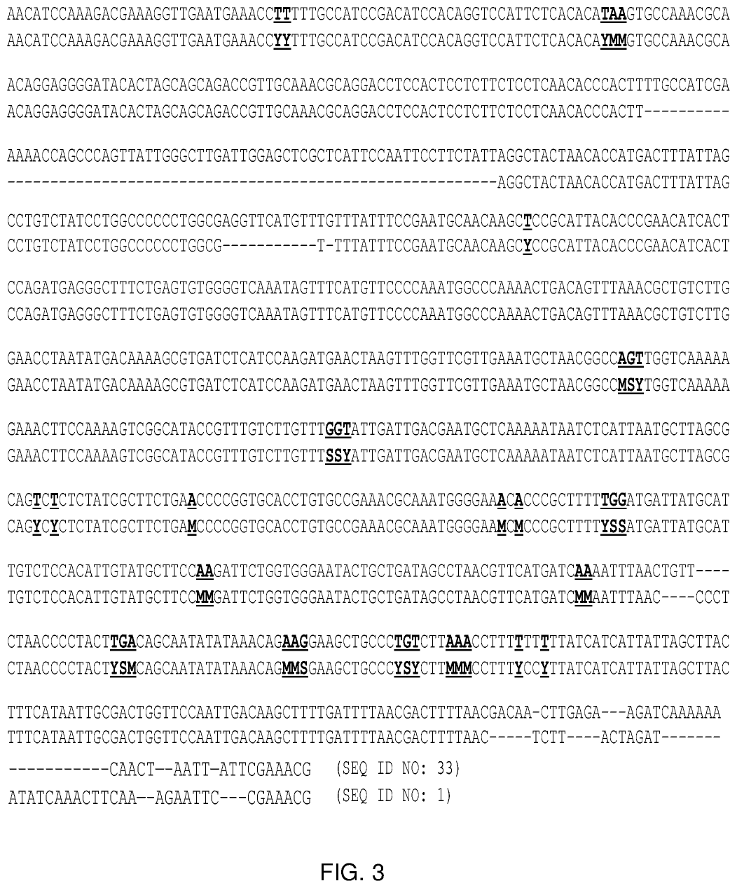

It will be appreciated that when a sequence of a first, shorter length is aligned with a sequence of a second, longer length, the resultant alignment may contain gaps in the first sequence that account for the relative difference in length between the two sequences. See, for example, the alignment as shown in FIG. 3. However, as used herein, a “sequence” is a contiguous chain of nucleotides having no spaces or gaps. An “aligned sequence” or the “alignment of” a sequence, relative to another sequence, may include gaps or spaces, as necessary for the alignment of interest.

The identity of related polynucleotide sequences can be readily calculated by any of the methods known to one of ordinary skill in the art. In preferred embodiments, the “percent identity” of two sequences (e.g., nucleic acid sequences) is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul et al., J. Mol. Biol. 215:403-10, 1990. Where gaps exist between two sequences, Gapped BLAST can be utilized, for example, as described in Altschul et al., Nucleic Acids Res. 25(17):3389-3402, 1997. When utilizing BLAST and Gapped BLAST programs, the default parameters of the respective programs (e.g., XBLAST and NBLAST) can be used, or the parameters can be adjusted appropriately as would be understood by one of ordinary skill in the art.

Another local alignment technique which may be used, for example, is based on the Smith-Waterman algorithm (Smith, T. F. & Waterman, M. S. (1981) “Identification of common molecular subsequences.” J. Mol. Biol. 147:195-197). A general global alignment technique which may be used, for example, is the Needleman-Wunsch algorithm (Needleman, S. B. & Wunsch, C. D. (1970) “A general method applicable to the search for similarities in the amino acid sequences of two proteins.” J. Mol. Biol. 48:443-453), which is based on dynamic programming.

More recently, a Fast Optimal Global Sequence Alignment Algorithm (FOGSAA) was developed that purportedly produces global alignment of nucleic acid and amino acid sequences faster than other optimal global alignment methods, including the Needleman-Wunsch algorithm. In some embodiments, the identity of two polynucleotides is determined by aligning the two nucleic acid sequences, calculating the number of identical nucleic acids, and dividing by the length of one of the nucleic acid sequences.

For multiple sequence alignments, computer programs including Clustal Omega (Sievers et al., Mol Syst Biol. 2011 Oct. 11; 7:539) may be used. In some embodiments, a nucleic acid sequence is found to have a specified percent identity to a reference sequence, such as a sequence disclosed in this application and/or recited in the claims, when sequence identity is determined using Clustal Omega (Sievers et al., Mol Syst Biol. 2011 Oct. 11; 7:539).

As used in this application, a residue (such as a nucleic acid residue) in sequence “X” is referred to as corresponding to a position or residue (such as a nucleic acid residue) “Z” in a different sequence “Y” when the residue in sequence X is at the counterpart position of Z in sequence Y when sequences X and Y are aligned using nucleic acid sequence alignment tools known in the art.

Mutations can be made in a nucleotide sequence by a variety of methods known to one of ordinary skill in the art. For example, mutations can be made by PCR-directed mutation, site-directed mutagenesis according to the method of Kunkel (Kunkel, Proc. Nat. Acad. Sci. U.S.A. 82: 488-492, 1985), by chemical synthesis of a gene encoding a polypeptide, by gene editing tools, or by insertions, such as insertion of a tag (e.g., a HIS tag or a GFP tag). Mutations can include, for example, substitutions, deletions, and translocations, generated by any method known in the art. Methods for producing mutations may be found in in references such as Molecular Cloning: A Laboratory Manual, J. Sambrook, et al., eds., Fourth Edition, Cold Spring Harbor Laboratory Press, Cold Spring Harbor, New York, 2012, or Current Protocols in Molecular Biology, F. M. Ausubel, et al., eds., John Wiley & Sons, Inc., New York, 2010.

In some embodiments, methods for producing variants include circular permutation (Yu and Lutz, Trends Biotechnol. 2011 January; 29(1):18-25). In circular permutation, the linear primary sequence of a polypeptide can be circularized (e.g., by joining the N-terminal and C-terminal ends of the sequence) and the polypeptide can be severed (“broken”) at a different location. Thus, the linear primary sequence of the new polypeptide may have low sequence identity (e.g., less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, or less than 5%, including all values in between) as determined by linear sequence alignment methods (e.g., Clustal Omega or BLAST). Topological analysis of the two proteins, however, may reveal that the tertiary structure of the two polypeptides is similar or dissimilar. Without being bound by a particular theory, a variant polypeptide created through circular permutation of a reference polypeptide and with a similar tertiary structure as the reference polypeptide can share similar functional characteristics (e.g., enzymatic activity, enzyme kinetics, substrate specificity, or product specificity). In some instances, circular permutation may alter the secondary structure, tertiary structure, or quaternary structure and produce an enzyme with different functional characteristics (e.g., increased or decreased enzymatic activity, different substrate specificity, or different product specificity). See, e.g., Yu and Lutz, Trends Biotechnol. 2011 Jan; 29(1):18-25.

It should be appreciated that, in a protein that has undergone circular permutation, the linear amino acid sequence of the protein would differ from a reference protein that has not undergone circular permutation. However, one of ordinary skill in the art would be able to determine which residues in the protein that has undergone circular permutation correspond to residues in the reference protein that has not undergone circular permutation by, for example, aligning the sequences and detecting conserved motifs, and/or by comparing the structures or predicted structures of the proteins, e.g., by homology modeling.

In some embodiments, variant sequences include homologous sequences. As used in this application, homologous sequences are sequences (e.g., nucleic acid sequences) that share a certain percent identity (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 86%, at least 87%, at least 88%, at least 89%, at least 90%, at least 95%, at least 96%, at least 97%, at least 98%, at least 99%, or 100% percent identity, including all values in between). Homologous sequences include but are not limited to paralogous sequences, orthologous sequences, or sequences arising from convergent evolution. In some embodiments, paralogous sequences arise from duplication of a gene within a genome of a species, while orthologous sequences diverge after a speciation event. Two different species may have evolved independently but may each comprise a sequence that shares a certain percent identity with a sequence from the other species as a result of convergent evolution.

In some embodiments, a polynucleotide variant comprises a domain that shares a secondary structure with a reference polynucleotide. In some embodiments, a polynucleotide variant shares a tertiary structure with a reference polynucleotide. As a non-limiting example, a variant polynucleotide may have low primary sequence identity (e.g., less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, or less than 5% sequence identity) compared to a reference polynucleotide, but share one or more secondary structures (e.g., double helices, stem-loop structure, etc.), or have the same tertiary structure as a reference polynucleotide (e.g., major and minor groove triplexes, etc.). Homology modeling may be used to compare two or more tertiary structures.

Functional variants of the proteins, enzymes, or other bioproducts disclosed in this application are also encompassed by this disclosure. Functional variants may be identified using any method known in the art. For example, the algorithm of Karlin and Altschul Proc. Natl. Acad. Sci. U.S.A. 87:2264-68, 1990 described above may be used to identify homologous proteins with known functions. Putative functional variants may also be identified by searching for polypeptides with functionally annotated domains. Databases including Pfam (Sonnhammer et al., Proteins. 1997 July; 28(3):405-20) may be used to identify polypeptides with a particular domain.

The skilled artisan will also realize that mutations in a bioproduct coding sequence may result in conservative amino acid substitutions to provide functionally equivalent variants of the foregoing bioproducts, e.g., variants that retain the activities of the bioproducts. As used in this application, a “conservative amino acid substitution” refers to an amino acid substitution that does not alter the relative charge or size characteristics or functional activity of the bioproduct in which the amino acid substitution is made.

The skilled artisan will also realize that mutations in a recombinant polypeptide coding sequence may result in conservative amino acid substitutions to provide functionally equivalent variants of the foregoing polypeptides, e.g., variants that retain the activities of the polypeptides. As used in this application, a “conservative amino acid substitution” refers to an amino acid substitution that does not alter the relative charge or size characteristics or functional activity of the protein in which the amino acid substitution is made.

In some instances, an amino acid is characterized by its R group (see, e.g., Table 2). For example, an amino acid may comprise a nonpolar aliphatic R group, a positively charged R group, a negatively charged R group, a nonpolar aromatic R group, or a polar uncharged R group. Non-limiting examples of an amino acid comprising a nonpolar aliphatic R group include alanine, glycine, valine, leucine, methionine, and isoleucine. Non-limiting examples of an amino acid comprising a positively charged R group include lysine, arginine, and histidine. Non-limiting examples of an amino acid comprising a negatively charged R group include aspartate and glutamate. Non-limiting examples of an amino acid comprising a nonpolar, aromatic R group include phenylalanine, tyrosine, and tryptophan. Non-limiting examples of an amino acid comprising a polar uncharged R group include serine, threonine, cysteine, proline, asparagine, and glutamine.

Non-limiting examples of functionally equivalent variants of polypeptides may include conservative amino acid substitutions in the amino acid sequences of proteins disclosed in this application. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D. Additional non-limiting examples of conservative amino acid substitutions are provided in Table 2.

In some embodiments, 1, 2, 3, 4, 5, 6, 7, 8, 9, 10, 11, 12, 13, 14, 15, 16, 17, 18, 19, 20 or more than 20 residues can be changed when preparing variant polypeptides. In some embodiments, amino acids are replaced by conservative amino acid substitutions.

TABLE 2
Non-limiting Examples of conservative amino acid substitutions.

| | | Conservative Amino |
|---|---|---|
| Original Residue | R Group Type | Acid Substitutions |
| Ala | nonpolar aliphatic R group | Cys, Gly, Ser |
| Arg | positively charged R group | His, Lys |
| Asn | polar uncharged R group | Asp, Gln, Glu |
| Asp | negatively charged R group | Asn, Gln, Glu |
| Cys | polar uncharged R group | Ala, Ser |
| Gln | polar uncharged R group | Asn, Asp, Glu |
| Glu | negatively charged R group | Asn, Asp, Gln |
| Gly | nonpolar aliphatic R group | Ala, Ser |
| His | positively charged R group | Arg, Tyr, Trp |
| Ile | nonpolar aliphatic R group | Leu, Met, Val |
| Leu | nonpolar aliphatic R group | Ile, Met, Val |
| Lys | positively charged R group | Arg, His |
| Met | nonpolar aliphatic R group | Ile, Leu, Phe, Val |
| Pro | polar uncharged R group | |
| Phe | nonpolar aromatic R group | Met, Trp, Tyr |
| Ser | polar uncharged R group | Ala, Gly, Thr |
| Thr | polar uncharged R group | Ala, Asn, Ser |
| Trp | nonpolar aromatic R group | His, Phe, Tyr, Met |
| Tyr | nonpolar aromatic R group | His, Phe, Trp |
| Val | nonpolar aliphatic R group | Ile, Leu, Met, Thr |

Amino acid substitutions in the amino acid sequence of a polypeptide to produce a recombinant polypeptide variant having a desired property and/or activity can be made by alteration of the coding sequence of the polypeptide. Similarly, conservative amino acid substitutions in the amino acid sequence of a polypeptide to produce functionally equivalent variants of the polypeptide typically are made by alteration of the coding sequence of the recombinant polypeptide.

In some embodiments, a polynucleotide encoding any of the bioproducts described in this application is under the control of one or more regulatory sequences. In some embodiments, a polynucleotide is expressed under the control of a promoter. In some embodiments, the promoter is a native promoter. As used herein, a “native” promoter refers to a promoter for which at least one copy naturally occurs in a host cell. A native promoter may include but is not limited to the original copy or copies in the host cell; a promoter at a different locus from its native locus in a cell is nonetheless considered a promoter that is native to the cell. In some embodiments, the promoter is synthetic.

The phraseology and terminology used in this application is for the purpose of description and should not be regarded as limiting. The use of terms such as “including,” “comprising,” “having,” “containing,” “involving,” and/or variations thereof in this application, is meant to encompass the items listed thereafter and equivalents thereof as well as additional items.

This invention is further illustrated by the Examples. Specific details of any particular method, process, medium, or condition in the Examples are examples only and not intended to be limiting.

### ENUMERATED EMBODIMENTS

Certain embodiments are set forth in the enumerated clauses below.
Clause 1. A synthetic promoter comprising a nucleic acid sequence as shown in SEQ ID NO: 1, wherein Y may be C or T, S may be G or C, and M may be A or C.
Clause 2. The synthetic promoter of clause 1, wherein (a) the nucleotide corresponding to position 32 is a C, or (b) the nucleotide corresponding to position 32 is a C, and all other Y bases in the sequence are T, all S bases in the sequence are G, and all M bases in the sequence are A.
Clause 3. The synthetic promoter of clause 1 or clause 2, wherein (a) the nucleotide to position 33 is a C, or (b) the nucleotide corresponding to position 33 is a C, and corresponding all other Y bases in the sequence are T, all S bases in the sequence are G, and all M bases in the sequence are A.
Clause 4. The synthetic promoter of any one of clauses 1-3, wherein (a) the nucleotide corresponding to position 70 is a C, or (b) the nucleotide corresponding to position 70 is a C, and all other Y bases in the sequence are T, all S bases in the sequence are G, and all M bases in the sequence are A.
Clause 5. The synthetic promoter of any one of clauses 1-4, wherein (a) the nucleotide corresponding to position 71 is a C, or (b) the nucleotide corresponding to position 71 is a C, and all Y bases in the sequence are T, all S bases in the sequence are G, and all other M bases in the sequence are A.
Clause 6. The synthetic promoter of any one of clauses 1-5, wherein (a) the nucleotide corresponding to position 72 is a C, or (b) the nucleotide corresponding to position 72 is a C, and all Y bases in the sequence are T, all S bases in the sequence are G, and all other M bases in the sequence are A.
Clause 7. The synthetic promoter of any one of clauses 1-6, wherein (a) the nucleotide corresponding to position 234 is a C, or (b) the nucleotide corresponding to position 234 is a C, and all Y bases in the sequence are T, all S bases in the sequence are G, and all other M bases in the sequence are A.
Clause 8. The synthetic promoter of any one of clauses 1-7, wherein (a) the nucleotide corresponding to position 413 is a C, or (b) the nucleotide corresponding to position 413 is a C, and all Y bases in the sequence are T, all S bases in the sequence are G, and all other M bases in the sequence are A.
Clause 9. The synthetic promoter of any one of clauses 1-8, wherein (a) the nucleotide corresponding to position 414 is a C, or (b) the nucleotide corresponding to position 414 is a C, and all Y bases in the sequence are T, all S bases in the sequence are G, and all other M bases in the sequence are A.
Clause 10. The synthetic promoter of any one of clauses 1-9, wherein (a) the nucleotide corresponding to position 415 is a C, or (b) the nucleotide corresponding to position 415 is a C, and all Y bases in the sequence are T, all S bases in the sequence are G, and all other M bases in the sequence are A.
Clause 11. The synthetic promoter of any one of clauses 1-10, wherein (a) the nucleotide corresponding to position 463 is a C, or (b) the nucleotide corresponding to position 463 is a C, and all Y bases in the sequence are T, all S bases in the sequence are G, and all other M bases in the sequence are A.
Clause 12. The synthetic promoter of any one of clauses 1-11, wherein (a) the nucleotide corresponding to position 464 is a C, or (b) the nucleotide corresponding to position 464 is a C, and all Y bases in the sequence are T, all S bases in the sequence are G, and all other M bases in the sequence are A.
Clause 13. The synthetic promoter of any one of clauses 1-12, wherein (a) the nucleotide corresponding to position 465 is a C, or (b) the nucleotide corresponding to position 465 is a C, and all Y bases in the sequence are T, all S bases in the sequence are G, and all other M bases in the sequence are A.
Clause 14. The synthetic promoter of any one of clauses 1-13, wherein (a) the nucleotide corresponding to position 513 is a C, or (b) the nucleotide corresponding to position 513 is a C, and all Y bases in the sequence are T, all S bases in the sequence are G, and all other M bases in the sequence are A.
Clause 15. The synthetic promoter of any one of clauses 1-14, wherein (a) the nucleotide corresponding to position 515 is a C, or (b) the nucleotide corresponding to position 515 is a C, and all Y bases in the sequence are T, all S bases in the sequence are G, and all other M bases in the sequence are A.
Clause 16. The synthetic promoter of any one of clauses 1-15, wherein (a) the nucleotide corresponding to position 531 is a C, or (b) the nucleotide corresponding to position 531 is a C, and all Y bases in the sequence are T, all S bases in the sequence are G, and all other M bases in the sequence are A.
Clause 17. The synthetic promoter of any one of clauses 1-16, wherein (a) the nucleotide corresponding to position 567 is a C, or (b) the nucleotide corresponding to position 567 is a C, and all Y bases in the sequence are T, all S bases in the sequence are G, and all other M bases in the sequence are A.
Clause 18. The synthetic promoter of any one of clauses 1-17, wherein (a) the nucleotide corresponding to position 569 is a C, or (b) the nucleotide corresponding to position 569 is a C, and all Y bases in the sequence are T, all S bases in the sequence are G, and all other M bases in the sequence are A.
Clause 19. The synthetic promoter of any one of clauses 1-18, wherein (a) the nucleotide corresponding to position 579 is a C, or (b) the nucleotide corresponding to position 579 is a C, and all Y bases in the sequence are T, all S bases in the sequence are G, and all other M bases in the sequence are A.
Clause 20. The synthetic promoter of any one of clauses 1-19, wherein (a) the nucleotide corresponding to position 580 is a C, or (b) the nucleotide corresponding to position 580 is a C, and all Y bases in the sequence are T, all S bases in the sequence are G, and all other M bases in the sequence are A.
Clause 21. The synthetic promoter of any one of clauses 1-20, wherein (a) the nucleotide corresponding to position 581 is a C, or (b) the nucleotide corresponding to position 581 is a C, and all Y bases in the sequence are T, all S bases in the sequence are G, and all other M bases in the sequence are A.
Clause 22. The synthetic promoter of any one of clauses 1-21, wherein (a) the nucleotide corresponding to position 616 is a C, or (b) the nucleotide corresponding to position 616 is a C, and all Y bases in the sequence are T, all S bases in the sequence are G, and all other M bases in the sequence are A.
Clause 23. The synthetic promoter of any one of clauses 1-22, wherein (a) the nucleotide corresponding to position 617 is a C, or (b) the nucleotide corresponding to position 617 is a C, and all Y bases in the sequence are T, all S bases in the sequence are G, and all other M bases in the sequence are A.
Clause 24. The synthetic promoter of any one of clauses 1-23, wherein (a) the nucleotide corresponding to position 660 is a C, or (b) the nucleotide corresponding to position 660 is a C, and all Y bases in the sequence are T, all S bases in the sequence are G, and all other M bases in the sequence are A.
Clause 25. The synthetic promoter of any one of clauses 1-24, wherein (a) the nucleotide corresponding to position 661 is a C, or (b) the nucleotide corresponding to position 661 is a C, and all Y bases in the sequence are T, all S bases in the sequence are G, and all other M bases in the sequence are A.
Clause 26. The synthetic promoter of any one of clauses 1-25, wherein (a) the nucleotide corresponding to position 686 is a C, or (b) the nucleotide corresponding to position 686 is a C, and all Y bases in the sequence are T, all S bases in the sequence are G, and all other M bases in the sequence are A.
Clause 27. The synthetic promoter of any one of clauses 1-26, wherein (a) the nucleotide corresponding to position 687 is a C, or (b) the nucleotide corresponding to position 687 is a C, and all Y bases in the sequence are T, all S bases in the sequence are G, and all other M bases in the sequence are A.
Clause 28. The synthetic promoter of any one of clauses 1-27, wherein (a) the nucleotide corresponding to position 688 is a C, or (b) the nucleotide corresponding to position 688 is a C, and all Y bases in the sequence are T, all S bases in the sequence are G, and all other M bases in the sequence are A.
Clause 29. The synthetic promoter of any one of clauses 1-28, wherein (a) the nucleotide corresponding to position 706 is a C, or (b) the nucleotide corresponding to position 706 is a C, and all Y bases in the sequence are T, all S bases in the sequence are G, and all other M bases in the sequence are A.
Clause 30. The synthetic promoter of any one of clauses 1-29, wherein (a) the nucleotide corresponding to position 707 is a C, or (b) the nucleotide corresponding to position 707 is a C, and all Y bases in the sequence are T, all S bases in the sequence are G, and all other M bases in the sequence are A.
Clause 31. The synthetic promoter of any one of clauses 1-30, wherein (a) the nucleotide corresponding to position 708 is a C, or (b) the nucleotide corresponding to position 708 is a C, and all Y bases in the sequence are T, all S bases in the sequence are G, and all other M bases in the sequence are A.
Clause 32. The synthetic promoter of any one of clauses 1-31, wherein (a) the nucleotide corresponding to position 719 is a C, or (b) the nucleotide corresponding to position 719 is a C, and all Y bases in the sequence are T, all S bases in the sequence are G, and all other M bases in the sequence are A.
Clause 33. The synthetic promoter of any one of clauses 1-32, wherein (a) the nucleotide corresponding to position 720 is a C, or (b) the nucleotide corresponding to position 720 is a C, and all Y bases in the sequence are T, all S bases in the sequence are G, and all other M bases in the sequence are A.
Clause 34. The synthetic promoter of any one of clauses 1-33, wherein (a) the nucleotide corresponding to position 721 is a C, or (b) the nucleotide corresponding to position 721 is a C, and all Y bases in the sequence are T, all S bases in the sequence are G, and all other M bases in the sequence are A.
Clause 35. The synthetic promoter of any one of clauses 1-34, wherein (a) the nucleotide corresponding to position 725 is a C, or (b) the nucleotide corresponding to position 725 is a C, and all Y bases in the sequence are T, all S bases in the sequence are G, and all other M bases in the sequence are A.
Clause 36. The synthetic promoter of any one of clauses 1-35, wherein (a) the nucleotide corresponding to position 726 is a C, or (b) the nucleotide corresponding to position 726 is a C, and all Y bases in the sequence are T, all S bases in the sequence are G, and all other M bases in the sequence are A.
Clause 37. The synthetic promoter of any one of clauses 1-36, wherein (a) the nucleotide corresponding to position 727 is a C, or (b) the nucleotide corresponding to position 727 is a C, and all Y bases in the sequence are T, all S bases in the sequence are G, and all other M bases in the sequence are A.
Clause 38. The synthetic promoter of any one of clauses 1-37, wherein (a) the nucleotide corresponding to position 733 is a C, or (b) the nucleotide corresponding to position 733 is a C, and all Y bases in the sequence are T, all S bases in the sequence are G, and all other M bases in the sequence are A.
Clause 39. The synthetic promoter of any one of clauses 1-38, wherein (a) the nucleotide corresponding to position 736 is a C, or (b) the nucleotide corresponding to position 736 is a C, and all Y bases in the sequence are T, all S bases in the sequence are G, and all other M bases in the sequence are A.
Clause 40. A synthetic promoter comprising one to thirty-eight bases different than SEQ ID NO: 33, wherein the one to thirty-eight bases that are different are located at position(s) 32, 33, 70, 71, 72, 313, 492, 493, 494, 542, 543, 544, 592, 594, 610, 646, 648, 658, 659, 660, 695, 696, 739, 740, 765, 766, 767, 785, 786, 787, 798, 799, 800, 804, 805, 806, 812, and/or 815 of SEQ ID NO: 33.
Clause 41. A synthetic promoter comprising a polynucleotide having at least 90%, at least 95%, or at least 99% identity to a nucleic acid sequence as shown in any one of SEQ ID NOs: 2-32.
Clause 42. A synthetic promoter comprising a polynucleotide having no more than 38 substitutions relative to a nucleic acid sequence as shown in any one of SEQ ID NOs: 2-32.
Clause 43. A synthetic promoter having a nucleic acid sequence as shown in any one of SEQ ID NOs: 2-32.

### EXAMPLES

### Example 1. Library Construction

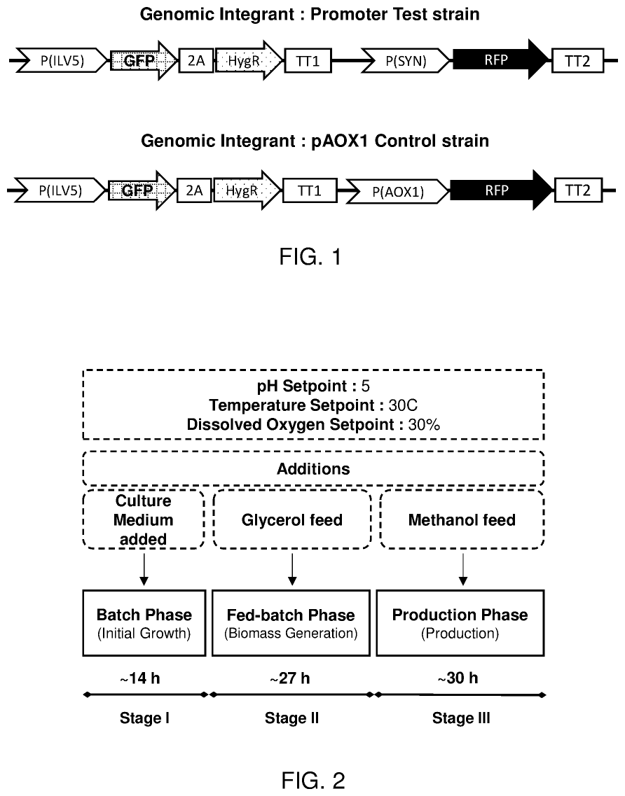

A library of synthetic promoters was generated, and promoters were tested as part of an integration vector and used to transform yeast host cells (FIG. 1) expressing red fluorescent protein (RFP). The synthetic promoters were integrated into the yeast host cells by homologous recombination, at single copy, in the locus corresponding to a native promoter of the yeast host cell, so that the native transcriptional terminator of the native yeast promoter could serve as the transcriptional terminator of the transcriptional unit expressing RFP. The correct integration of the synthetic promoter in each resulting strain was clonally verified by next generation sequencing (NGS). Strains were cryo-preserved in 30% glycerol.

TABLE 3
Differences between consensus sequence (SEQ ID NO: 1) and different synthetic promoters. Numbers in the
column for Promoter (e.g., 4168032) indicate molecule identification numbers for different synthetic
promoters. For the consensus sequence, abbreviations (e.g., Y, M, and S) are as described in Table 1.

| | POSITION | | | | | | | | | | |
|---|---|---|---|---|---|---|---|---|---|---|---|
| | | 70, | | 413, | 463, | | | | | | 579, |
| Promoter | 32, 33 | 71, 72 | 234 | 414, 415 | 444, 465 | 513 | 515 | 531 | 567 | 569 | 580, 581 |
| Consensus | YY | YMM | Y | MSY | SSY | Y | Y | M | M | M | YSS |
| 4168032 | TT | CCC | T | AGT | GGT | C | C | A | A | A | TGG |
| 4168038 | TT | CCC | C | AGT | GGT | T | T | A | A | A | TGG |
| 4168039 | CC | TAA | T | CCC | GGT | T | T | A | A | A | TGG |
| 4168041 | CC | TAA | T | AGT | GGT | T | T | A | A | A | TGG |
| 4168045 | TT | TAA | C | AGT | GGT | T | T | A | A | A | CCC |
| 4168047 | TT | TAA | T | AGT | GGT | T | T | C | A | A | TGG |
| 4168051 | TT | TAA | T | AGT | GGT | T | T | A | C | C | TGG |
| 4168052 | TT | TAA | T | AGT | CCC | T | T | C | A | A | TGG |
| 4168054 | TT | CCC | T | AGT | GGT | C | C | A | A | A | TGG |
| 4168057 | TT | CCC | T | AGT | GGT | T | T | A | A | A | TGG |
| 4168059 | TT | TAA | T | AGT | GGT | C | C | A | A | A | TGG |
| 4168061 | CC | TAA | T | AGT | CCC | T | T | A | A | A | TGG |
| 4168067 | CC | TAA | T | AGT | GGT | T | T | A | C | C | TGG |
| 4168070 | CC | TAA | T | AGT | GGT | T | T | A | A | A | TGG |
| 4168073 | TT | TAA | C | AGT | CCC | T | T | A | A | A | TGG |
| 4168076 | TT | TAA | T | AGT | GGT | T | T | C | A | A | TGG |
| 4168078 | CC | TAA | T | AGT | GGT | T | T | A | A | A | TGG |
| 4168084 | TT | CCC | T | AGT | GGT | T | T | A | A | A | TGG |
| 4168085 | TT | CCC | T | AGT | GGT | T | T | C | A | A | TGG |
| 4168090 | TT | TAA | T | AGT | GGT | T | T | C | A | A | CCC |
| 4168092 | TT | CCC | T | AGT | GGT | T | T | A | C | C | TGG |
| 4168094 | TT | TAA | T | AGT | CCC | T | T | C | A | A | TGG |
| 4168095 | TT | TAA | T | AGT | GGT | T | T | A | C | C | TGG |
| 4168097 | TT | TAA | T | AGT | GGT | C | C | A | A | A | TGG |
| 4168098 | TT | TAA | C | AGT | GGT | T | T | A | A | A | TGG |
| 4168101 | TT | TAA | T | CCC | GGT | T | T | C | A | A | TGG |
| 4168104 | TT | TAA | T | CCC | GGT | T | T | C | A | A | TGG |

| | POSITION | | | | | | | |
|---|---|---|---|---|---|---|---|---|
| | | | 686, | 706, | 719, | 725, | | |
| Promoter | 616, 617 | 660, 661 | 687, 688 | 707, 708 | 720, 721 | 726, 727 | 733 | 736 |
| Consensus | MM | MM | YSM | MMS | YSY | MMM | Y | Y |
| 4168032 | AA | AA | TGA | CCC | TGT | AAA | T | C |
| 4168038 | AA | AA | TGA | AAG | CCC | AAA | T | C |
| 4168039 | AA | AA | TGA | CCC | TGT | CCC | C | T |
| 4168041 | AA | AA | TGA | AAG | CCC | AAA | T | C |
| 4168045 | AA | AA | TGA | AAG | CCC | AAA | T | C |
| 4168047 | AA | AA | CCC | CCC | TGT | AAA | T | C |
| 4168051 | CC | CC | TGA | AAG | CCC | AAA | T | C |
| 4168052 | AA | AA | TGA | CCC | TGT | CCC | C | T |
| 4168054 | AA | AA | TGA | AAG | CCC | AAA | T | C |
| 4168057 | AA | AA | TGA | CCC | TGT | AAA | T | C |
| 4168059 | CC | CC | TGA | AAG | CCC | AAA | T | C |
| 4168061 | AA | AA | TGA | CCC | TGT | CCC | C | T |
| 4168067 | AA | AA | TGA | CCC | TGT | CCC | C | T |
| 4168070 | CC | CC | TGA | CCC | TGT | CCC | C | T |
| 4168073 | AA | AA | TGA | CCC | TGT | CCC | C | T |
| 4168076 | AA | AA | CCC | AAG | CCC | AAA | T | C |
| 4168078 | AA | AA | CCC | AAG | CCC | AAA | T | C |
| 4168084 | CC | CC | TGA | CCC | TGT | AAA | T | C |
| 4168085 | AA | AA | TGA | CCC | TGT | AAA | T | C |
| 4168090 | AA | AA | TGA | CCC | TGT | AAA | T | C |
| 4168092 | AA | AA | TGA | CCC | TGT | AAA | T | C |
| 4168094 | AA | AA | TGA | CCC | TGT | AAA | T | C |
| 4168095 | CC | CC | TGA | CCC | TGT | AAA | T | C |
| 4168097 | AA | AA | TGA | AAG | CCC | AAA | T | C |
| 4168098 | AA | AA | CCC | CCC | TGT | AAA | T | C |
| 4168101 | AA | AA | TGA | CCC | TGT | CCC | C | T |
| 4168104 | AA | AA | TGA | CCC | TGT | AAA | T | C |

### Example 2. Assay of Promoter Strength in Deepwell Plates

A glycerol stock of each member of the library of transformed yeast strains was spotted onto a yeast extract peptone (YEP)+4% dextrose agar plate and allowed to grow at 30° C. for 48 hours. These colonies were used to inoculate 200 μl of YEP+2% dextrose liquid media in a deepwell plate and allowed to grow at 30° C. for 24 hours. These cultures were then subcultured in 200 μl of BMY medium (Buffered Minimal medium with Yeast extract, a buffered complex yeast growth media), supplemented with 1% glycerol, and grown at 30° C. for 24 hours. Cells were then washed twice with phosphate-buffered saline (PBS). Cell density and intracellular fluorescence were measured in a plate reader using a small aliquot. Fluorescence readings were normalized to cell density and represented the pre-induction activity at this stage. The washed cells were resuspended in 200 μl of BMY medium supplemented with 1% methanol and grown at 30° C. for 24 hours. Cells were then washed with PBS. Cell density and intracellular fluorescence were taken again, as described before, with normalized fluorescence units representing the post-induction activity. Control strains were tested and evaluated in an equivalent manner alongside test strains. Results are shown in Table 4.

Composition of deepwell plate culture media: BMY medium contains yeast extract, peptone, yeast nitrogen base (without amino acids), potassium phosphate, and biotin, while YEP medium contains yeast extract, bacto peptone, and NaCl.

TABLE 4
Promoter activity in a deepwell plate assay, as measured via fluorescence
activity of red fluorescence protein (RFP). Promoters in bold
type (e.g., 4168032, 4168061, etc.) were subjected to a lab scale
methanol-based fermentation (see Examples 3 and 4).

| | Median Specific | | Percent of | |
|---|---|---|---|---|
| | Fluorescence | | P(AOX1) | Fold Induction |
| | Pre- | Post- | activity post- | (Post/Pre- |
| Promoter ID | induction | induction | induction | induction) |
| P(AOX1) | 1335 | 32313 | 100.00% | 24.20 |
| 4168024 | 3992 | 28584 | 88.46% | 7.16 |
| 4168032 | 3628 | 56642 | 175.29% | 15.61 |
| 4168038 | 6278 | 53384 | 165.21% | 8.50 |
| 4168039 | 3780 | 36423 | 112.72% | 9.64 |
| 4168041 | 2798 | 35838 | 110.91% | 12.81 |
| 4168043 | 3745 | 6600 | 20.43% | 1.76 |
| 4168045 | 6660 | 65351 | 202.24% | 9.81 |
| 4168047 | 4052 | 61587 | 190.60% | 15.20 |
| 4168051 | 8241 | 73062 | 226.11% | 8.87 |
| 4168052 | 4279 | 38931 | 120.48% | 9.10 |
| 4168054 | 3907 | 47925 | 148.31% | 12.27 |
| 4168057 | 3579 | 33853 | 104.77% | 9.46 |
| 4168059 | 4296 | 38603 | 119.47% | 8.99 |
| 4168061 | 3200 | 34912 | 108.04% | 10.91 |
| 4168067 | 6164 | 61840 | 191.38% | 10.03 |
| 4168070 | 3853 | 43784 | 135.50% | 11.36 |
| 4168072 | 5250 | 27691 | 85.70% | 5.27 |
| 4168073 | 6301 | 63759 | 197.32% | 10.12 |
| 4168076 | 4169 | 51538 | 159.50% | 12.36 |
| 4168078 | 2862 | 69920 | 216.38% | 24.43 |
| 4168079 | 5910 | 9424 | 29.16% | 1.59 |
| 4168084 | 6880 | 44780 | 138.58% | 6.51 |
| 4168085 | 3646 | 45683 | 141.38% | 12.53 |
| 4168090 | 4417 | 62165 | 192.38% | 14.07 |
| 4168092 | 6444 | 58087 | 179.76% | 9.01 |
| 4168094 | 3765 | 62244 | 192.63% | 16.53 |
| 4168095 | 2024 | 44129 | 136.57% | 21.80 |
| 4168097 | 2974 | 47442 | 146.82% | 15.95 |
| 4168098 | 4057 | 59804 | 185.08% | 14.74 |
| 4168101 | 4621 | 53393 | 165.24% | 11.55 |
| 4168104 | 3647 | 57030 | 176.49% | 15.64 |

### Example 3: Fermentation Process

Freshly grown colonies of the strain(s) of interest were scraped from a solid culture medium plate and used to inoculate an erlenmeyer shake flask with culture medium supplemented with glycerol. Alternately, the shake flask could be directly inoculated with a thawed glycerol stock of the strain(s). The culture was allowed to grow for 18-20 hours at 30° C., 250 rpm to an optical density (OD) at 600 nm of 20±5. This served as an inoculum for a bioreactor, which was prefilled with fresh culture medium. Glycerol was added to a final concentration of 40 g/L. The bioreactor operated continuously while maintaining constant pH, temperature, and dissolved oxygen levels (FIG. 2), with no additional glycerol being fed during the batch phase. The end of the batch phase was marked by a complete consumption of added glycerol, and a glycerol feed was initiated to mark the fed-batch phase. The fed-batch phase ended when sufficient biomass was achieved, and the production phase was initiated by transitioning to a methanol feed. Fermentation ended 86 to 96 hours after the start of fermentation.

Composition of Culture medium: Potassium phosphate monobasic, Ammonium sulfate, Calcium sulfate dihydrate, Potassium sulfate, Magnesium sulfate heptahydrate, Copper (II) Sulfate Pentahydrate, Sodium Iodide, Manganese (II) Sulfate Monohydrate, Sodium Molybdate Dihydrate, Boric Acid, Calcium Sulfate Dihydrate, Cobalt (II) Chloride Zinc Chloride, Iron (II) Sulfate Heptahydrate, Biotin, and Sulfuric Acid.

### Example 4. Assay of Promoter Strength in Lab-Scale Bioreactors

A subset of strains from Example 2 was subjected to a lab scale methanol-based fermentation using the process described in Example 3. Samples were drawn after the start of fermentation every 12 hours, until 48 hours had elapsed, and then every 6 hours thereafter, until hours had elapsed, and were stored at 4° C. after a 100-fold dilution in PBS. Each sample was subjected to flow cytometry, and the median fluorescence value of 100,000 cells was measured. Table 5 summarizes the performance of library members in comparison to the P(AOX1) control strain. The sequences for the various synthetic promoters and the control promoter are in Table 6.

TABLE 5
Promoter activity in a lab-scale fermentation assay, as measured
via fluorescence activity of red fluorescence protein.

| Time | 4168078 | 4168032 | 4168067 | 4168092 | 4168094 | 4168095 | 4168097 | 4168061 | pAOX1 |
|---|---|---|---|---|---|---|---|---|---|
| 0 h | 1901 | 3525 | 3386 | 5576 | 3927 | 3289 | 1337 | 1905 | 1339 |
| 12 h | 150 | 189 | 203 | 238 | 199 | 191 | 137 | 146 | 131 |
| 24 h | 2400 | 2807 | 4316 | 5364 | 2963 | 1013 | 2576 | 2630 | 1180 |
| 36 h | 3397 | 4339 | 11043 | 11834 | 4109 | 693 | 3555 | 3582 | 1428 |
| 48 h | 4254 | 6206 | 7290 | 9961 | 7122 | 2862 | 6383 | 6369 | 3344 |
| 54 h | 13111 | 14659 | 15765 | 18372 | 15109 | 7276 | 13201 | 13269 | 8171 |
| 60 h | 19529 | 20993 | 22085 | 24973 | 21169 | 10568 | 18529 | 18466 | 11614 |
| 66 h | 25197 | 26313 | 26950 | 30319 | 25879 | 13293 | 22497 | 23004 | 14052 |
| 72 h | 25839 | 26456 | 25792 | 27868 | 24640 | 13008 | 22118 | 22081 | 13846 |
| 78 h | 27824 | 29769 | 27386 | 32183 | 28389 | 15115 | 25229 | 24242 | 15448 |
| 84 h | 31635 | 32239 | 31285 | 30990 | 27821 | 15553 | 26675 | 26019 | 16494 |
| 90 h | 32682 | 33097 | 34717 | 37194 | 33673 | 17883 | 28274 | 27359 | 17240 |

TABLE 6
Nucleotide sequences of synthetic promoters and control promoter
[P(AOX1)]. For the consensus sequence, abbreviations (e.g., Y, M, and S)
are as described in Table 1.

| | |
|---|---|
| Consensus | AACATCCAAAGACGAAAGGTTGAATGAAACCYYTTTGCCATCCGACATCCAC |
| sequence | AGGTCCATTCTCACACAYMMGTGCCAAACGCAACAGGAGGGGATACACTAGC |
| | AGCAGACCGTTGCAAACGCAGGACCTCCACTCCTCTTCTCCTCAACACCCAC |
| | TTAGGCTACTAACACCATGACTTTATTAGCCTGTCTATCCTGGCCCCCCTGG |
| | CGTTTTATTTCCGAATGCAACAAGCYCCGCATTACACCCGAACATCACTCCA |
| | GATGAGGGCTTTCTGAGTGTGGGGTCAAATAGTTTCATGTTCCCCAAATGGC |
| | CCAAAACTGACAGTTTAAACGCTGTCTTGGAACCTAATATGACAAAAGCGTG |
| | ATCTCATCCAAGATGAACTAAGTTTGGTTCGTTGAAATGCTAACGGCCMSYT |
| | GGTCAAAAAGAAACTTCCAAAAGTCGGCATACCGTTTGTCTTGTTTSSYATT |
| | GATTGACGAATGCTCAAAAATAATCTCATTAATGCTTAGCGCAGYCYCTCTA |
| | TCGCTTCTGAMCCCCGGTGCACCTGTGCCGAAACGCAAATGGGGAAMCMCCC |
| | GCTTTTYSSATGATTATGCATTGTCTCCACATTGTATGCTTCCMMGATTCTG |
| | GTGGGAATACTGCTGATAGCCTAACGTTCATGATCMMAATTTAACCCCTCTA |
| | ACCCCTACTYSMCAGCAATATATAAACAGMMSGAAGCTGCCCYSYCTTMMMC |
| | CTTTYCCYTTATCATCATTATTAGCTTACTTTCATAATTGCGACTGGTTCCA |
| | ATTGACAAGCTTTTGATTTTAACGACTTTTAACTCTTACTAGATATATCAAA |
| | CTTCAAAGAATTCCGAAACG (SEQ ID NO: 1) |
| Synthetic | AACATCCAAAGACGAAAGGTTGAATGAAACCTTTTTGCCATCCGACATCCAC |
| MeOH- | AGGTCCATTCTCACACATAAGTGCCAAACGCAACAGGAGGGGATACACTAGC |
| inducible | AGCAGACCGTTGCAAACGCAGGACCTCCACTCCTCTTCTCCTCAACACCCAC |
| promoter | TTAGGCTACTAACACCATGACTTTATTAGCCTGTCTATCCTGGCCCCCCTGG |
| (4168024) | CGTTTTATTTCCGAATGCAACAAGCTCCGCATTACACCCGAACATCACTCCA |
| | GATGAGGGCTTTCTGAGTGTGGGGTCAAATAGTTTCATGTTCCCCAAATGGC |
| | CCAAAACTGACAGTTTAAACGCTGTCTTGGAACCTAATATGACAAAAGCGTG |
| | ATCTCATCCAAGATGAACTAAGTTTGGTTCGTTGAAATGCTAACGGCCAGTT |
| | GGTCAAAAAGAAACTTCCAAAAGTCGGCATACCGTTTGTCTTGTTTGGTATT |
| | GATTGACGAATGCTCAAAAATAATCTCATTAATGCTTAGCGCAGTCTCTCTA |
| | TCGCTTCTGAACCCCGGTGCACCTGTGCCGAAACGCAAATGGGGAACCCCCC |
| | GCTTTTTGGATGATTATGCATTGTCTCCACATTGTATGCTTCCAAGATTCTG |
| | GTGGGAATACTGCTGATAGCCTAACGTTCATGATCCCAATTTAACCCCTCTA |
| | ACCCCTACTTGACAGCAATATATAAACAGCCCGAAGCTGCCCTGTCTTCCCC |
| | CTTTCCCTTTATCATCATTATTAGCTTACTTTCATAATTGCGACTGGTTCCA |
| | ATTGACAAGCTTTTGATTTTAACGACTTTTAACTCTTACTAGATATATCAAA |
| | CTTCAAAGAATTCCGAAACG (SEQ ID NO: 2) |
| Synthetic | AACATCCAAAGACGAAAGGTTGAATGAAACCTTTTTGCCATCCGACATCCAC |
| MeOH- | AGGTCCATTCTCACACACCCGTGCCAAACGCAACAGGAGGGGATACACTAGC |
| inducible | AGCAGACCGTTGCAAACGCAGGACCTCCACTCCTCTTCTCCTCAACACCCAC |
| promoter | TTAGGCTACTAACACCATGACTTTATTAGCCTGTCTATCCTGGCCCCCCTGG |
| (4168032) | CGTTTTATTTCCGAATGCAACAAGCTCCGCATTACACCCGAACATCACTCCA |
| | GATGAGGGCTTTCTGAGTGTGGGGTCAAATAGTTTCATGTTCCCCAAATGGC |
| | CCAAAACTGACAGTTTAAACGCTGTCTTGGAACCTAATATGACAAAAGCGTG |
| | ATCTCATCCAAGATGAACTAAGTTTGGTTCGTTGAAATGCTAACGGCCAGTT |
| | GGTCAAAAAGAAACTTCCAAAAGTCGGCATACCGTTTGTCTTGTTTGGTATT |
| | GATTGACGAATGCTCAAAAATAATCTCATTAATGCTTAGCGCAGCCCCTCTA |
| | TCGCTTCTGAACCCCGGTGCACCTGTGCCGAAACGCAAATGGGGAAACACCC |
| | GCTTTTTGGATGATTATGCATTGTCTCCACATTGTATGCTTCCAAGATTCTG |
| | GTGGGAATACTGCTGATAGCCTAACGTTCATGATCAAAATTTAACCCCTCTA |
| | ACCCCTACTTGACAGCAATATATAAACAGCCCGAAGCTGCCCTGTCTTAAAC |
| | CTTTTCCCTTATCATCATTATTAGCTTACTTTCATAATTGCGACTGGTTCCA |
| | ATTGACAAGCTTTTGATTTTAACGACTTTTAACTCTTACTAGATATATCAAA |
| | CTTCAAAGAATTCCGAAACG (SEQ ID NO: 3) |
| Synthetic | AACATCCAAAGACGAAAGGTTGAATGAAACCTTTTTGCCATCCGACATCCAC |
| MeOH- | AGGTCCATTCTCACACACCCGTGCCAAACGCAACAGGAGGGGATACACTAGC |
| inducible | AGCAGACCGTTGCAAACGCAGGACCTCCACTCCTCTTCTCCTCAACACCCAC |
| promoter | TTAGGCTACTAACACCATGACTTTATTAGCCTGTCTATCCTGGCCCCCCTGG |
| (4168038) | CGTTTTATTTCCGAATGCAACAAGCCCCGCATTACACCCGAACATCACTCCA |
| | GATGAGGGCTTTCTGAGTGTGGGGTCAAATAGTTTCATGTTCCCCAAATGGC |
| | CCAAAACTGACAGTTTAAACGCTGTCTTGGAACCTAATATGACAAAAGCGTG |
| | ATCTCATCCAAGATGAACTAAGTTTGGTTCGTTGAAATGCTAACGGCCAGTT |
| | GGTCAAAAAGAAACTTCCAAAAGTCGGCATACCGTTTGTCTTGTTTGGTATT |
| | GATTGACGAATGCTCAAAAATAATCTCATTAATGCTTAGCGCAGTCTCTCTA |
| | TCGCTTCTGAACCCCGGTGCACCTGTGCCGAAACGCAAATGGGGAAACACCC |
| | GCTTTTTGGATGATTATGCATTGTCTCCACATTGTATGCTTCCAAGATTCTG |
| | GTGGGAATACTGCTGATAGCCTAACGTTCATGATCAAAATTTAACCCCTCTA |
| | ACCCCTACTTGACAGCAATATATAAACAGAAGGAAGCTGCCCCCCCTTAAAC |
| | CTTTTCCCTTATCATCATTATTAGCTTACTTTCATAATTGCGACTGGTTCCA |
| | ATTGACAAGCTTTTGATTTTAACGACTTTTAACTCTTACTAGATATATCAAA |
| | CTTCAAAGAATTCCGAAACG (SEQ ID NO: 4) |
| Synthetic | AACATCCAAAGACGAAAGGTTGAATGAAACCCCTTTGCCATCCGACATCCAC |
| MeOH- | AGGTCCATTCTCACACATAAGTGCCAAACGCAACAGGAGGGGATACACTAGC |
| inducible | AGCAGACCGTTGCAAACGCAGGACCTCCACTCCTCTTCTCCTCAACACCCAC |
| promoter | TTAGGCTACTAACACCATGACTTTATTAGCCTGTCTATCCTGGCCCCCCTGG |
| (4168039) | CGTTTTATTTCCGAATGCAACAAGCTCCGCATTACACCCGAACATCACTCCA |
| | GATGAGGGCTTTCTGAGTGTGGGGTCAAATAGTTTCATGTTCCCCAAATGGC |
| | CCAAAACTGACAGTTTAAACGCTGTCTTGGAACCTAATATGACAAAAGCGTG |
| | ATCTCATCCAAGATGAACTAAGTTTGGTTCGTTGAAATGCTAACGGCCCCCT |
| | GGTCAAAAAGAAACTTCCAAAAGTCGGCATACCGTTTGTCTTGTTTGGTATT |
| | GATTGACGAATGCTCAAAAATAATCTCATTAATGCTTAGCGCAGTCTCTCTA |
| | TCGCTTCTGAACCCCGGTGCACCTGTGCCGAAACGCAAATGGGGAAACACCC |
| | GCTTTTTGGATGATTATGCATTGTCTCCACATTGTATGCTTCCAAGATTCTG |
| | GTGGGAATACTGCTGATAGCCTAACGTTCATGATCAAAATTTAACCCCTCTA |
| | ACCCCTACTTGACAGCAATATATAAACAGCCCGAAGCTGCCCTGTCTTCCCC |
| | CTTTCCCTTTATCATCATTATTAGCTTACTTTCATAATTGCGACTGGTTCCA |
| | ATTGACAAGCTTTTGATTTTAACGACTTTTAACTCTTACTAGATATATCAAA |
| | CTTCAAAGAATTCCGAAACG (SEQ ID NO: 5) |
| Synthetic | AACATCCAAAGACGAAAGGTTGAATGAAACCCCTTTGCCATCCGACATCCAC |
| MeOH- | AGGTCCATTCTCACACATAAGTGCCAAACGCAACAGGAGGGGATACACTAGC |
| inducible | AGCAGACCGTTGCAAACGCAGGACCTCCACTCCTCTTCTCCTCAACACCCAC |
| promoter | TTAGGCTACTAACACCATGACTTTATTAGCCTGTCTATCCTGGCCCCCCTGG |
| (4168041) | CGTTTTATTTCCGAATGCAACAAGCTCCGCATTACACCCGAACATCACTCCA |
| | GATGAGGGCTTTCTGAGTGTGGGGTCAAATAGTTTCATGTTCCCCAAATGGC |
| | CCAAAACTGACAGTTTAAACGCTGTCTTGGAACCTAATATGACAAAAGCGTG |
| | ATCTCATCCAAGATGAACTAAGTTTGGTTCGTTGAAATGCTAACGGCCAGTT |
| | GGTCAAAAAGAAACTTCCAAAAGTCGGCATACCGTTTGTCTTGTTTGGTATT |
| | GATTGACGAATGCTCAAAAATAATCTCATTAATGCTTAGCGCAGTCTCTCTA |
| | TCGCTTCTGAACCCCGGTGCACCTGTGCCGAAACGCAAATGGGGAAACACCC |
| | GCTTTTTGGATGATTATGCATTGTCTCCACATTGTATGCTTCCAAGATTCTG |
| | GTGGGAATACTGCTGATAGCCTAACGTTCATGATCCCAATTTAACCCCTCTA |
| | ACCCCTACTTGACAGCAATATATAAACAGAAGGAAGCTGCCCCCCCTTAAAC |
| | CTTTTCCCTTATCATCATTATTAGCTTACTTTCATAATTGCGACTGGTTCCA |
| | ATTGACAAGCTTTTGATTTTAACGACTTTTAACTCTTACTAGATATATCAAA |
| | CTTCAAAGAATTCCGAAACG (SEQ ID NO: 6) |
| Synthetic | AACATCCAAAGACGAAAGGTTGAATGAAACCCCTTTGCCATCCGACATCCAC |
| MeOH- | AGGTCCATTCTCACACATAAGTGCCAAACGCAACAGGAGGGGATACACTAGC |
| inducible | AGCAGACCGTTGCAAACGCAGGACCTCCACTCCTCTTCTCCTCAACACCCAC |
| promoter | TTAGGCTACTAACACCATGACTTTATTAGCCTGTCTATCCTGGCCCCCCTGG |
| (4168043) | CGTTTTATTTCCGAATGCAACAAGCTCCGCATTACACCCGAACATCACTCCA |
| | GATGAGGGCTTTCTGAGTGTGGGGTCAAATAGTTTCATGTTCCCCAAATGGC |
| | CCAAAACTGACAGTTTAAACGCTGTCTTGGAACCTAATATGACAAAAGCGTG |
| | ATCTCATCCAAGATGAACTAAGTTTGGTTCGTTGAAATGCTAACGGCCAGTT |
| | GGTCAAAAAGAAACTTCCAAAAGTCGGCATACCGTTTGTCTTGTTTGGTATT |
| | GATTGACGAATGCTCAAAAATAATCTCATTAATGCTTAGCGCAGCCCCTCTA |
| | TCGCTTCTGAACCCCGGTGCACCTGTGCCGAAACGCAAATGGGGAAACACCC |
| | GCTTTTTGGATGATTATGCATTGTCTCCACATTGTATGCTTCCAAGATTCTG |
| | GTGGGAATACTGCTGATAGCCTAACGTTCATGATCAAAATTTAACCCCTCTA |
| | ACCCCTACTTGACAGCAATATATAAACAGCCCGAAGCTGCCCTGTCTTAAAC |
| | CTTTTCCCTTATCATCATTATTAGCTTACTTTCATAATTGCGACTGGTTCCA |
| | ATTGACAAGCTTTTGATTTTAACGACTTTTAACTCTTACTAGATATATCAAA |
| | CTTCAAAGAATTCCGAAACG (SEQ ID NO: 7) |
| Synthetic | AACATCCAAAGACGAAAGGTTGAATGAAACCTTTTTGCCATCCGACATCCAC |
| MeOH- | AGGTCCATTCTCACACATAAGTGCCAAACGCAACAGGAGGGGATACACTAGC |
| inducible | AGCAGACCGTTGCAAACGCAGGACCTCCACTCCTCTTCTCCTCAACACCCAC |
| promoter | TTAGGCTACTAACACCATGACTTTATTAGCCTGTCTATCCTGGCCCCCCTGG |
| (4168045) | CGTTTTATTTCCGAATGCAACAAGCCCCGCATTACACCCGAACATCACTCCA |
| | GATGAGGGCTTTCTGAGTGTGGGGTCAAATAGTTTCATGTTCCCCAAATGGC |
| | CCAAAACTGACAGTTTAAACGCTGTCTTGGAACCTAATATGACAAAAGCGTG |
| | ATCTCATCCAAGATGAACTAAGTTTGGTTCGTTGAAATGCTAACGGCCAGTT |
| | GGTCAAAAAGAAACTTCCAAAAGTCGGCATACCGTTTGTCTTGTTTGGTATT |
| | GATTGACGAATGCTCAAAAATAATCTCATTAATGCTTAGCGCAGTCTCTCTA |
| | TCGCTTCTGAACCCCGGTGCACCTGTGCCGAAACGCAAATGGGGAAACACCC |
| | GCTTTTCCCATGATTATGCATTGTCTCCACATTGTATGCTTCCAAGATTCTG |
| | GTGGGAATACTGCTGATAGCCTAACGTTCATGATCAAAATTTAACCCCTCTA |
| | ACCCCTACTTGACAGCAATATATAAACAGAAGGAAGCTGCCCCCCCTTAAAC |
| | CTTTTCCCTTATCATCATTATTAGCTTACTTTCATAATTGCGACTGGTTCCA |
| | ATTGACAAGCTTTTGATTTTAACGACTTTTAACTCTTACTAGATATATCAAA |
| | CTTCAAAGAATTCCGAAACG (SEQ ID NO: 8) |
| Synthetic | AACATCCAAAGACGAAAGGTTGAATGAAACCTTTTTGCCATCCGACATCCAC |
| MeOH- | AGGTCCATTCTCACACATAAGTGCCAAACGCAACAGGAGGGGATACACTAGC |
| inducible | AGCAGACCGTTGCAAACGCAGGACCTCCACTCCTCTTCTCCTCAACACCCAC |
| promoter | TTAGGCTACTAACACCATGACTTTATTAGCCTGTCTATCCTGGCCCCCCTGG |
| (4168047) | CGTTTTATTTCCGAATGCAACAAGCTCCGCATTACACCCGAACATCACTCCA |
| | GATGAGGGCTTTCTGAGTGTGGGGTCAAATAGTTTCATGTTCCCCAAATGGC |
| | CCAAAACTGACAGTTTAAACGCTGTCTTGGAACCTAATATGACAAAAGCGTG |
| | ATCTCATCCAAGATGAACTAAGTTTGGTTCGTTGAAATGCTAACGGCCAGTT |
| | GGTCAAAAAGAAACTTCCAAAAGTCGGCATACCGTTTGTCTTGTTTGGTATT |
| | GATTGACGAATGCTCAAAAATAATCTCATTAATGCTTAGCGCAGTCTCTCTA |
| | TCGCTTCTGACCCCCGGTGCACCTGTGCCGAAACGCAAATGGGGAAACACCC |
| | GCTTTTTGGATGATTATGCATTGTCTCCACATTGTATGCTTCCAAGATTCTG |
| | GTGGGAATACTGCTGATAGCCTAACGTTCATGATCAAAATTTAACCCCTCTA |
| | ACCCCTACTCCCCAGCAATATATAAACAGCCCGAAGCTGCCCTGTCTTAAAC |
| | CTTTTCCCTTATCATCATTATTAGCTTACTTTCATAATTGCGACTGGTTCCA |
| | ATTGACAAGCTTTTGATTTTAACGACTTTTAACTCTTACTAGATATATCAAA |
| | CTTCAAAGAATTCCGAAACG (SEQ ID NO: 9) |
| Synthetic | AACATCCAAAGACGAAAGGTTGAATGAAACCTTTTTGCCATCCGACATCCAC |
| MeOH- | AGGTCCATTCTCACACATAAGTGCCAAACGCAACAGGAGGGGATACACTAGC |
| inducible | AGCAGACCGTTGCAAACGCAGGACCTCCACTCCTCTTCTCCTCAACACCCAC |
| promoter | TTAGGCTACTAACACCATGACTTTATTAGCCTGTCTATCCTGGCCCCCCTGG |
| (4168051) | CGTTTTATTTCCGAATGCAACAAGCTCCGCATTACACCCGAACATCACTCCA |
| | GATGAGGGCTTTCTGAGTGTGGGGTCAAATAGTTTCATGTTCCCCAAATGGC |
| | CCAAAACTGACAGTTTAAACGCTGTCTTGGAACCTAATATGACAAAAGCGTG |
| | ATCTCATCCAAGATGAACTAAGTTTGGTTCGTTGAAATGCTAACGGCCAGTT |
| | GGTCAAAAAGAAACTTCCAAAAGTCGGCATACCGTTTGTCTTGTTTGGTATT |
| | GATTGACGAATGCTCAAAAATAATCTCATTAATGCTTAGCGCAGTCTCTCTA |
| | TCGCTTCTGAACCCCGGTGCACCTGTGCCGAAACGCAAATGGGGAACCCCCC |
| | GCTTTTTGGATGATTATGCATTGTCTCCACATTGTATGCTTCCCCGATTCTG |
| | GTGGGAATACTGCTGATAGCCTAACGTTCATGATCAAAATTTAACCCCTCTA |
| | ACCCCTACTTGACAGCAATATATAAACAGAAGGAAGCTGCCCCCCCTTAAAC |
| | CTTTTCCCTTATCATCATTATTAGCTTACTTTCATAATTGCGACTGGTTCCA |
| | ATTGACAAGCTTTTGATTTTAACGACTTTTAACTCTTACTAGATATATCAAA |
| | CTTCAAAGAATTCCGAAACG (SEQ ID NO: 10) |
| Synthetic | AACATCCAAAGACGAAAGGTTGAATGAAACCTTTTTGCCATCCGACATCCAC |
| MeOH- | AGGTCCATTCTCACACATAAGTGCCAAACGCAACAGGAGGGGATACACTAGC |
| inducible | AGCAGACCGTTGCAAACGCAGGACCTCCACTCCTCTTCTCCTCAACACCCAC |
| promoter | TTAGGCTACTAACACCATGACTTTATTAGCCTGTCTATCCTGGCCCCCCTGG |
| (4168052) | CGTTTTATTTCCGAATGCAACAAGCTCCGCATTACACCCGAACATCACTCCA |
| | GATGAGGGCTTTCTGAGTGTGGGGTCAAATAGTTTCATGTTCCCCAAATGGC |
| | CCAAAACTGACAGTTTAAACGCTGTCTTGGAACCTAATATGACAAAAGCGTG |
| | ATCTCATCCAAGATGAACTAAGTTTGGTTCGTTGAAATGCTAACGGCCAGTT |
| | GGTCAAAAAGAAACTTCCAAAAGTCGGCATACCGTTTGTCTTGTTTCCCATT |
| | GATTGACGAATGCTCAAAAATAATCTCATTAATGCTTAGCGCAGTCTCTCTA |
| | TCGCTTCTGACCCCCGGTGCACCTGTGCCGAAACGCAAATGGGGAAACACCC |
| | GCTTTTTGGATGATTATGCATTGTCTCCACATTGTATGCTTCCAAGATTCTG |
| | GTGGGAATACTGCTGATAGCCTAACGTTCATGATCAAAATTTAACCCCTCTA |
| | ACCCCTACTTGACAGCAATATATAAACAGCCCGAAGCTGCCCTGTCTTCCCC |
| | CTTTCCCTTTATCATCATTATTAGCTTACTTTCATAATTGCGACTGGTTCCA |
| | ATTGACAAGCTTTTGATTTTAACGACTTTTAACTCTTACTAGATATATCAAA |
| | CTTCAAAGAATTCCGAAACG (SEQ ID NO: 11) |
| Synthetic | AACATCCAAAGACGAAAGGTTGAATGAAACCTTTTTGCCATCCGACATCCAC |
| MeOH- | AGGTCCATTCTCACACACCCGTGCCAAACGCAACAGGAGGGGATACACTAGC |
| inducible | AGCAGACCGTTGCAAACGCAGGACCTCCACTCCTCTTCTCCTCAACACCCAC |
| promoter | TTAGGCTACTAACACCATGACTTTATTAGCCTGTCTATCCTGGCCCCCCTGG |
| (4168054) | CGTTTTATTTCCGAATGCAACAAGCTCCGCATTACACCCGAACATCACTCCA |
| | GATGAGGGCTTTCTGAGTGTGGGGTCAAATAGTTTCATGTTCCCCAAATGGC |
| | CCAAAACTGACAGTTTAAACGCTGTCTTGGAACCTAATATGACAAAAGCGTG |
| | ATCTCATCCAAGATGAACTAAGTTTGGTTCGTTGAAATGCTAACGGCCAGTT |
| | GGTCAAAAAGAAACTTCCAAAAGTCGGCATACCGTTTGTCTTGTTTGGTATT |
| | GATTGACGAATGCTCAAAAATAATCTCATTAATGCTTAGCGCAGCCCCTCTA |
| | TCGCTTCTGAACCCCGGTGCACCTGTGCCGAAACGCAAATGGGGAAACACCC |
| | GCTTTTTGGATGATTATGCATTGTCTCCACATTGTATGCTTCCAAGATTCTG |
| | GTGGGAATACTGCTGATAGCCTAACGTTCATGATCAAAATTTAACCCCTCTA |
| | ACCCCTACTTGACAGCAATATATAAACAGAAGGAAGCTGCCCCCCCTTAAAC |
| | CTTTTCCCTTATCATCATTATTAGCTTACTTTCATAATTGCGACTGGTTCCA |
| | ATTGACAAGCTTTTGATTTTAACGACTTTTAACTCTTACTAGATATATCAAA |
| | CTTCAAAGAATTCCGAAACG (SEQ ID NO: 12) |
| Synthetic | AACATCCAAAGACGAAAGGTTGAATGAAACCTTTTTGCCATCCGACATCCAC |
| MeOH- | AGGTCCATTCTCACACACCCGTGCCAAACGCAACAGGAGGGGATACACTAGC |
| inducible | AGCAGACCGTTGCAAACGCAGGACCTCCACTCCTCTTCTCCTCAACACCCAC |
| promoter | TTAGGCTACTAACACCATGACTTTATTAGCCTGTCTATCCTGGCCCCCCTGG |
| (4168057) | CGTTTTATTTCCGAATGCAACAAGCTCCGCATTACACCCGAACATCACTCCA |
| | GATGAGGGCTTTCTGAGTGTGGGGTCAAATAGTTTCATGTTCCCCAAATGGC |
| | CCAAAACTGACAGTTTAAACGCTGTCTTGGAACCTAATATGACAAAAGCGTG |
| | ATCTCATCCAAGATGAACTAAGTTTGGTTCGTTGAAATGCTAACGGCCAGTT |
| | GGTCAAAAAGAAACTTCCAAAAGTCGGCATACCGTTTGTCTTGTTTGGTATT |
| | GATTGACGAATGCTCAAAAATAATCTCATTAATGCTTAGCGCAGTCTCTCTA |
| | TCGCTTCTGAACCCCGGTGCACCTGTGCCGAAACGCAAATGGGGAAACACCC |
| | GCTTTTTGGATGATTATGCATTGTCTCCACATTGTATGCTTCCAAGATTCTG |
| | GTGGGAATACTGCTGATAGCCTAACGTTCATGATCCCAATTTAACCCCTCTA |
| | ACCCCTACTTGACAGCAATATATAAACAGCCCGAAGCTGCCCTGTCTTAAAC |
| | CTTTTCCCTTATCATCATTATTAGCTTACTTTCATAATTGCGACTGGTTCCA |
| | ATTGACAAGCTTTTGATTTTAACGACTTTTAACTCTTACTAGATATATCAAA |
| | CTTCAAAGAATTCCGAAACG (SEQ ID NO: 13) |
| Synthetic | AACATCCAAAGACGAAAGGTTGAATGAAACCTTTTTGCCATCCGACATCCAC |
| MeOH- | AGGTCCATTCTCACACATAAGTGCCAAACGCAACAGGAGGGGATACACTAGC |
| inducible | AGCAGACCGTTGCAAACGCAGGACCTCCACTCCTCTTCTCCTCAACACCCAC |
| promoter | TTAGGCTACTAACACCATGACTTTATTAGCCTGTCTATCCTGGCCCCCCTGG |
| (4168059) | CGTTTTATTTCCGAATGCAACAAGCTCCGCATTACACCCGAACATCACTCCA |
| | GATGAGGGCTTTCTGAGTGTGGGGTCAAATAGTTTCATGTTCCCCAAATGGC |
| | CCAAAACTGACAGTTTAAACGCTGTCTTGGAACCTAATATGACAAAAGCGTG |
| | ATCTCATCCAAGATGAACTAAGTTTGGTTCGTTGAAATGCTAACGGCCAGTT |
| | GGTCAAAAAGAAACTTCCAAAAGTCGGCATACCGTTTGTCTTGTTTGGTATT |
| | GATTGACGAATGCTCAAAAATAATCTCATTAATGCTTAGCGCAGCCCCTCTA |
| | TCGCTTCTGAACCCCGGTGCACCTGTGCCGAAACGCAAATGGGGAAACACCC |
| | GCTTTTTGGATGATTATGCATTGTCTCCACATTGTATGCTTCCCCGATTCTG |
| | GTGGGAATACTGCTGATAGCCTAACGTTCATGATCAAAATTTAACCCCTCTA |
| | ACCCCTACTTGACAGCAATATATAAACAGAAGGAAGCTGCCCCCCCTTAAAC |
| | CTTTTCCCTTATCATCATTATTAGCTTACTTTCATAATTGCGACTGGTTCCA |
| | ATTGACAAGCTTTTGATTTTAACGACTTTTAACTCTTACTAGATATATCAAA |
| | CTTCAAAGAATTCCGAAACG (SEQ ID NO: 14) |
| Synthetic | AACATCCAAAGACGAAAGGTTGAATGAAACCCCTTTGCCATCCGACATCCAC |
| MeOH- | AGGTCCATTCTCACACATAAGTGCCAAACGCAACAGGAGGGGATACACTAGC |
| inducible | AGCAGACCGTTGCAAACGCAGGACCTCCACTCCTCTTCTCCTCAACACCCAC |
| promoter | TTAGGCTACTAACACCATGACTTTATTAGCCTGTCTATCCTGGCCCCCCTGG |
| (4168061) | CGTTTTATTTCCGAATGCAACAAGCTCCGCATTACACCCGAACATCACTCCA |
| | GATGAGGGCTTTCTGAGTGTGGGGTCAAATAGTTTCATGTTCCCCAAATGGC |
| | CCAAAACTGACAGTTTAAACGCTGTCTTGGAACCTAATATGACAAAAGCGTG |
| | ATCTCATCCAAGATGAACTAAGTTTGGTTCGTTGAAATGCTAACGGCCAGTT |
| | GGTCAAAAAGAAACTTCCAAAAGTCGGCATACCGTTTGTCTTGTTTCCCATT |
| | GATTGACGAATGCTCAAAAATAATCTCATTAATGCTTAGCGCAGTCTCTCTA |
| | TCGCTTCTGAACCCCGGTGCACCTGTGCCGAAACGCAAATGGGGAAACACCC |
| | GCTTTTTGGATGATTATGCATTGTCTCCACATTGTATGCTTCCAAGATTCTG |
| | GTGGGAATACTGCTGATAGCCTAACGTTCATGATCAAAATTTAACCCCTCTA |
| | ACCCCTACTTGACAGCAATATATAAACAGCCCGAAGCTGCCCTGTCTTCCCC |
| | CTTTCCCTTTATCATCATTATTAGCTTACTTTCATAATTGCGACTGGTTCCA |
| | ATTGACAAGCTTTTGATTTTAACGACTTTTAACTCTTACTAGATATATCAAA |
| | CTTCAAAGAATTCCGAAACG (SEQ ID NO: 15) |
| Synthetic | AACATCCAAAGACGAAAGGTTGAATGAAACCCCTTTGCCATCCGACATCCAC |
| MeOH- | AGGTCCATTCTCACACATAAGTGCCAAACGCAACAGGAGGGGATACACTAGC |
| inducible | AGCAGACCGTTGCAAACGCAGGACCTCCACTCCTCTTCTCCTCAACACCCAC |
| promoter | TTAGGCTACTAACACCATGACTTTATTAGCCTGTCTATCCTGGCCCCCCTGG |
| (4168067) | CGTTTTATTTCCGAATGCAACAAGCTCCGCATTACACCCGAACATCACTCCA |
| | GATGAGGGCTTTCTGAGTGTGGGGTCAAATAGTTTCATGTTCCCCAAATGGC |
| | CCAAAACTGACAGTTTAAACGCTGTCTTGGAACCTAATATGACAAAAGCGTG |
| | ATCTCATCCAAGATGAACTAAGTTTGGTTCGTTGAAATGCTAACGGCCAGTT |
| | GGTCAAAAAGAAACTTCCAAAAGTCGGCATACCGTTTGTCTTGTTTGGTATT |
| | GATTGACGAATGCTCAAAAATAATCTCATTAATGCTTAGCGCAGTCTCTCTA |
| | TCGCTTCTGAACCCCGGTGCACCTGTGCCGAAACGCAAATGGGGAACCCCCC |
| | GCTTTTTGGATGATTATGCATTGTCTCCACATTGTATGCTTCCAAGATTCTG |
| | GTGGGAATACTGCTGATAGCCTAACGTTCATGATCAAAATTTAACCCCTCTA |
| | ACCCCTACTTGACAGCAATATATAAACAGCCCGAAGCTGCCCTGTCTTCCCC |
| | CTTTCCCTTTATCATCATTATTAGCTTACTTTCATAATTGCGACTGGTTCCA |
| | ATTGACAAGCTTTTGATTTTAACGACTTTTAACTCTTACTAGATATATCAAA |
| | CTTCAAAGAATTCCGAAACG (SEQ ID NO: 16) |
| Synthetic | AACATCCAAAGACGAAAGGTTGAATGAAACCCCTTTGCCATCCGACATCCAC |
| MeOH- | AGGTCCATTCTCACACATAAGTGCCAAACGCAACAGGAGGGGATACACTAGC |
| inducible | AGCAGACCGTTGCAAACGCAGGACCTCCACTCCTCTTCTCCTCAACACCCAC |
| promoter | TTAGGCTACTAACACCATGACTTTATTAGCCTGTCTATCCTGGCCCCCCTGG |
| (4168070) | CGTTTTATTTCCGAATGCAACAAGCTCCGCATTACACCCGAACATCACTCCA |
| | GATGAGGGCTTTCTGAGTGTGGGGTCAAATAGTTTCATGTTCCCCAAATGGC |
| | CCAAAACTGACAGTTTAAACGCTGTCTTGGAACCTAATATGACAAAAGCGTG |
| | ATCTCATCCAAGATGAACTAAGTTTGGTTCGTTGAAATGCTAACGGCCAGTT |
| | GGTCAAAAAGAAACTTCCAAAAGTCGGCATACCGTTTGTCTTGTTTGGTATT |
| | GATTGACGAATGCTCAAAAATAATCTCATTAATGCTTAGCGCAGTCTCTCTA |
| | TCGCTTCTGAACCCCGGTGCACCTGTGCCGAAACGCAAATGGGGAAACACCC |
| | GCTTTTTGGATGATTATGCATTGTCTCCACATTGTATGCTTCCCCGATTCTG |
| | GTGGGAATACTGCTGATAGCCTAACGTTCATGATCAAAATTTAACCCCTCTA |
| | ACCCCTACTTGACAGCAATATATAAACAGCCCGAAGCTGCCCTGTCTTCCCC |
| | CTTTCCCTTTATCATCATTATTAGCTTACTTTCATAATTGCGACTGGTTCCA |
| | ATTGACAAGCTTTTGATTTTAACGACTTTTAACTCTTACTAGATATATCAAA |
| | CTTCAAAGAATTCCGAAACG (SEQ ID NO: 17) |
| Synthetic | AACATCCAAAGACGAAAGGTTGAATGAAACCCCTTTGCCATCCGACATCCAC |
| MeOH- | AGGTCCATTCTCACACATAAGTGCCAAACGCAACAGGAGGGGATACACTAGC |
| inducible | AGCAGACCGTTGCAAACGCAGGACCTCCACTCCTCTTCTCCTCAACACCCAC |
| promoter | TTAGGCTACTAACACCATGACTTTATTAGCCTGTCTATCCTGGCCCCCCTGG |
| (4168072) | CGTTTTATTTCCGAATGCAACAAGCTCCGCATTACACCCGAACATCACTCCA |
| | GATGAGGGCTTTCTGAGTGTGGGGTCAAATAGTTTCATGTTCCCCAAATGGC |
| | CCAAAACTGACAGTTTAAACGCTGTCTTGGAACCTAATATGACAAAAGCGTG |
| | ATCTCATCCAAGATGAACTAAGTTTGGTTCGTTGAAATGCTAACGGCCAGTT |
| | GGTCAAAAAGAAACTTCCAAAAGTCGGCATACCGTTTGTCTTGTTTGGTATT |
| | GATTGACGAATGCTCAAAAATAATCTCATTAATGCTTAGCGCAGTCTCTCTA |
| | TCGCTTCTGAACCCCGGTGCACCTGTGCCGAAACGCAAATGGGGAAACACCC |
| | GCTTTTTGGATGATTATGCATTGTCTCCACATTGTATGCTTCCCCGATTCTG |
| | GTGGGAATACTGCTGATAGCCTAACGTTCATGATCAAAATTTAACCCCTCTA |
| | ACCCCTACTTGACAGCAATATATAAACAGAAGGAAGCTGCCCCCCCTTAAAC |
| | CTTTTCCCTTATCATCATTATTAGCTTACTTTCATAATTGCGACTGGTTCCA |
| | ATTGACAAGCTTTTGATTTTAACGACTTTTAACTCTTACTAGATATATCAAA |
| | CTTCAAAGAATTCCGAAACG (SEQ ID NO: 18) |
| Synthetic | AACATCCAAAGACGAAAGGTTGAATGAAACCTTTTTGCCATCCGACATCCAC |
| MeOH- | AGGTCCATTCTCACACATAAGTGCCAAACGCAACAGGAGGGGATACACTAGC |
| inducible | AGCAGACCGTTGCAAACGCAGGACCTCCACTCCTCTTCTCCTCAACACCCAC |
| promoter | TTAGGCTACTAACACCATGACTTTATTAGCCTGTCTATCCTGGCCCCCCTGG |
| (4168073) | CGTTTTATTTCCGAATGCAACAAGCCCCGCATTACACCCGAACATCACTCCA |
| | GATGAGGGCTTTCTGAGTGTGGGGTCAAATAGTTTCATGTTCCCCAAATGGC |
| | CCAAAACTGACAGTTTAAACGCTGTCTTGGAACCTAATATGACAAAAGCGTG |
| | ATCTCATCCAAGATGAACTAAGTTTGGTTCGTTGAAATGCTAACGGCCAGTT |
| | GGTCAAAAAGAAACTTCCAAAAGTCGGCATACCGTTTGTCTTGTTTCCCATT |
| | GATTGACGAATGCTCAAAAATAATCTCATTAATGCTTAGCGCAGTCTCTCTA |
| | TCGCTTCTGAACCCCGGTGCACCTGTGCCGAAACGCAAATGGGGAAACACCC |
| | GCTTTTTGGATGATTATGCATTGTCTCCACATTGTATGCTTCCAAGATTCTG |
| | GTGGGAATACTGCTGATAGCCTAACGTTCATGATCAAAATTTAACCCCTCTA |
| | ACCCCTACTTGACAGCAATATATAAACAGCCCGAAGCTGCCCTGTCTTCCCC |
| | CTTTCCCTTTATCATCATTATTAGCTTACTTTCATAATTGCGACTGGTTCCA |
| | ATTGACAAGCTTTTGATTTTAACGACTTTTAACTCTTACTAGATATATCAAA |
| | CTTCAAAGAATTCCGAAACG (SEQ ID NO: 19) |
| Synthetic | AACATCCAAAGACGAAAGGTTGAATGAAACCTTTTTGCCATCCGACATCCAC |
| MeOH- | AGGTCCATTCTCACACATAAGTGCCAAACGCAACAGGAGGGGATACACTAGC |
| inducible | AGCAGACCGTTGCAAACGCAGGACCTCCACTCCTCTTCTCCTCAACACCCAC |
| promoter | TTAGGCTACTAACACCATGACTTTATTAGCCTGTCTATCCTGGCCCCCCTGG |
| (4168076) | CGTTTTATTTCCGAATGCAACAAGCTCCGCATTACACCCGAACATCACTCCA |
| | GATGAGGGCTTTCTGAGTGTGGGGTCAAATAGTTTCATGTTCCCCAAATGGC |
| | CCAAAACTGACAGTTTAAACGCTGTCTTGGAACCTAATATGACAAAAGCGTG |
| | ATCTCATCCAAGATGAACTAAGTTTGGTTCGTTGAAATGCTAACGGCCAGTT |
| | GGTCAAAAAGAAACTTCCAAAAGTCGGCATACCGTTTGTCTTGTTTGGTATT |
| | GATTGACGAATGCTCAAAAATAATCTCATTAATGCTTAGCGCAGTCTCTCTA |
| | TCGCTTCTGACCCCCGGTGCACCTGTGCCGAAACGCAAATGGGGAAACACCC |
| | GCTTTTTGGATGATTATGCATTGTCTCCACATTGTATGCTTCCAAGATTCTG |
| | GTGGGAATACTGCTGATAGCCTAACGTTCATGATCAAAATTTAACCCCTCTA |
| | ACCCCTACTCCCCAGCAATATATAAACAGAAGGAAGCTGCCCCCCCTTAAAC |
| | CTTTTCCCTTATCATCATTATTAGCTTACTTTCATAATTGCGACTGGTTCCA |
| | ATTGACAAGCTTTTGATTTTAACGACTTTTAACTCTTACTAGATATATCAAA |
| | CTTCAAAGAATTCCGAAACG (SEQ ID NO: 20) |
| Synthetic | AACATCCAAAGACGAAAGGTTGAATGAAACCCCTTTGCCATCCGACATCCAC |
| MeOH- | AGGTCCATTCTCACACATAAGTGCCAAACGCAACAGGAGGGGATACACTAGC |
| inducible | AGCAGACCGTTGCAAACGCAGGACCTCCACTCCTCTTCTCCTCAACACCCAC |
| promoter | TTAGGCTACTAACACCATGACTTTATTAGCCTGTCTATCCTGGCCCCCCTGG |
| (4168078) | CGTTTTATTTCCGAATGCAACAAGCTCCGCATTACACCCGAACATCACTCCA |
| | GATGAGGGCTTTCTGAGTGTGGGGTCAAATAGTTTCATGTTCCCCAAATGGC |
| | CCAAAACTGACAGTTTAAACGCTGTCTTGGAACCTAATATGACAAAAGCGTG |
| | ATCTCATCCAAGATGAACTAAGTTTGGTTCGTTGAAATGCTAACGGCCAGTT |
| | GGTCAAAAAGAAACTTCCAAAAGTCGGCATACCGTTTGTCTTGTTTGGTATT |
| | GATTGACGAATGCTCAAAAATAATCTCATTAATGCTTAGCGCAGTCTCTCTA |
| | TCGCTTCTGAACCCCGGTGCACCTGTGCCGAAACGCAAATGGGGAAACACCC |
| | GCTTTTTGGATGATTATGCATTGTCTCCACATTGTATGCTTCCAAGATTCTG |
| | GTGGGAATACTGCTGATAGCCTAACGTTCATGATCAAAATTTAACCCCTCTA |
| | ACCCCTACTCCCCAGCAATATATAAACAGAAGGAAGCTGCCCCCCCTTAAAC |
| | CTTTTCCCTTATCATCATTATTAGCTTACTTTCATAATTGCGACTGGTTCCA |
| | ATTGACAAGCTTTTGATTTTAACGACTTTTAACTCTTACTAGATATATCAAA |
| | CTTCAAAGAATTCCGAAACG (SEQ ID NO: 21) |
| Synthetic | AACATCCAAAGACGAAAGGTTGAATGAAACCTTTTTGCCATCCGACATCCAC |
| MeOH- | AGGTCCATTCTCACACATAAGTGCCAAACGCAACAGGAGGGGATACACTAGC |
| inducible | AGCAGACCGTTGCAAACGCAGGACCTCCACTCCTCTTCTCCTCAACACCCAC |
| promoter | TTAGGCTACTAACACCATGACTTTATTAGCCTGTCTATCCTGGCCCCCCTGG |
| (4168079) | CGTTTTATTTCCGAATGCAACAAGCTCCGCATTACACCCGAACATCACTCCA |
| | GATGAGGGCTTTCTGAGTGTGGGGTCAAATAGTTTCATGTTCCCCAAATGGC |
| | CCAAAACTGACAGTTTAAACGCTGTCTTGGAACCTAATATGACAAAAGCGTG |
| | ATCTCATCCAAGATGAACTAAGTTTGGTTCGTTGAAATGCTAACGGCCAGTT |
| | GGTCAAAAAGAAACTTCCAAAAGTCGGCATACCGTTTGTCTTGTTTGGTATT |
| | GATTGACGAATGCTCAAAAATAATCTCATTAATGCTTAGCGCAGCCCCTCTA |
| | TCGCTTCTGAACCCCGGTGCACCTGTGCCGAAACGCAAATGGGGAAACACCC |
| | GCTTTTTGGATGATTATGCATTGTCTCCACATTGTATGCTTCCCCGATTCTG |
| | GTGGGAATACTGCTGATAGCCTAACGTTCATGATCAAAATTTAACCCCTCTA |
| | ACCCCTACTTGACAGCAATATATAAACAGCCCGAAGCTGCCCTGTCTTAAAC |
| | CTTTTCCCTTATCATCATTATTAGCTTACTTTCATAATTGCGACTGGTTCCA |
| | ATTGACAAGCTTTTGATTTTAACGACTTTTAACTCTTACTAGATATATCAAA |
| | CTTCAAAGAATTCCGAAACG (SEQ ID NO: 22) |
| Synthetic | AACATCCAAAGACGAAAGGTTGAATGAAACCTTTTTGCCATCCGACATCCAC |
| MeOH- | AGGTCCATTCTCACACACCCGTGCCAAACGCAACAGGAGGGGATACACTAGC |
| inducible | AGCAGACCGTTGCAAACGCAGGACCTCCACTCCTCTTCTCCTCAACACCCAC |
| promoter | TTAGGCTACTAACACCATGACTTTATTAGCCTGTCTATCCTGGCCCCCCTGG |
| (4168084) | CGTTTTATTTCCGAATGCAACAAGCTCCGCATTACACCCGAACATCACTCCA |
| | GATGAGGGCTTTCTGAGTGTGGGGTCAAATAGTTTCATGTTCCCCAAATGGC |
| | CCAAAACTGACAGTTTAAACGCTGTCTTGGAACCTAATATGACAAAAGCGTG |
| | ATCTCATCCAAGATGAACTAAGTTTGGTTCGTTGAAATGCTAACGGCCAGTT |
| | GGTCAAAAAGAAACTTCCAAAAGTCGGCATACCGTTTGTCTTGTTTGGTATT |
| | GATTGACGAATGCTCAAAAATAATCTCATTAATGCTTAGCGCAGTCTCTCTA |
| | TCGCTTCTGAACCCCGGTGCACCTGTGCCGAAACGCAAATGGGGAAACACCC |
| | GCTTTTTGGATGATTATGCATTGTCTCCACATTGTATGCTTCCCCGATTCTG |
| | GTGGGAATACTGCTGATAGCCTAACGTTCATGATCAAAATTTAACCCCTCTA |
| | ACCCCTACTTGACAGCAATATATAAACAGCCCGAAGCTGCCCTGTCTTAAAC |
| | CTTTTCCCTTATCATCATTATTAGCTTACTTTCATAATTGCGACTGGTTCCA |
| | ATTGACAAGCTTTTGATTTTAACGACTTTTAACTCTTACTAGATATATCAAA |
| | CTTCAAAGAATTCCGAAACG (SEQ ID NO: 23) |
| Synthetic | AACATCCAAAGACGAAAGGTTGAATGAAACCTTTTTGCCATCCGACATCCAC |
| MeOH- | AGGTCCATTCTCACACACCCGTGCCAAACGCAACAGGAGGGGATACACTAGC |
| inducible | AGCAGACCGTTGCAAACGCAGGACCTCCACTCCTCTTCTCCTCAACACCCAC |
| promoter | TTAGGCTACTAACACCATGACTTTATTAGCCTGTCTATCCTGGCCCCCCTGG |
| (4168085) | CGTTTTATTTCCGAATGCAACAAGCTCCGCATTACACCCGAACATCACTCCA |
| | GATGAGGGCTTTCTGAGTGTGGGGTCAAATAGTTTCATGTTCCCCAAATGGC |
| | CCAAAACTGACAGTTTAAACGCTGTCTTGGAACCTAATATGACAAAAGCGTG |
| | ATCTCATCCAAGATGAACTAAGTTTGGTTCGTTGAAATGCTAACGGCCAGTT |
| | GGTCAAAAAGAAACTTCCAAAAGTCGGCATACCGTTTGTCTTGTTTGGTATT |
| | GATTGACGAATGCTCAAAAATAATCTCATTAATGCTTAGCGCAGTCTCTCTA |
| | TCGCTTCTGACCCCCGGTGCACCTGTGCCGAAACGCAAATGGGGAAACACCC |
| | GCTTTTTGGATGATTATGCATTGTCTCCACATTGTATGCTTCCAAGATTCTG |
| | GTGGGAATACTGCTGATAGCCTAACGTTCATGATCAAAATTTAACCCCTCTA |
| | ACCCCTACTTGACAGCAATATATAAACAGCCCGAAGCTGCCCTGTCTTAAAC |
| | CTTTTCCCTTATCATCATTATTAGCTTACTTTCATAATTGCGACTGGTTCCA |
| | ATTGACAAGCTTTTGATTTTAACGACTTTTAACTCTTACTAGATATATCAAA |
| | CTTCAAAGAATTCCGAAACG (SEQ ID NO: 24) |
| Synthetic | AACATCCAAAGACGAAAGGTTGAATGAAACCTTTTTGCCATCCGACATCCAC |
| MeOH- | AGGTCCATTCTCACACATAAGTGCCAAACGCAACAGGAGGGGATACACTAGC |
| inducible | AGCAGACCGTTGCAAACGCAGGACCTCCACTCCTCTTCTCCTCAACACCCAC |
| promoter | TTAGGCTACTAACACCATGACTTTATTAGCCTGTCTATCCTGGCCCCCCTGG |
| (4168090) | CGTTTTATTTCCGAATGCAACAAGCTCCGCATTACACCCGAACATCACTCCA |
| | GATGAGGGCTTTCTGAGTGTGGGGTCAAATAGTTTCATGTTCCCCAAATGGC |
| | CCAAAACTGACAGTTTAAACGCTGTCTTGGAACCTAATATGACAAAAGCGTG |
| | ATCTCATCCAAGATGAACTAAGTTTGGTTCGTTGAAATGCTAACGGCCAGTT |
| | GGTCAAAAAGAAACTTCCAAAAGTCGGCATACCGTTTGTCTTGTTTGGTATT |
| | GATTGACGAATGCTCAAAAATAATCTCATTAATGCTTAGCGCAGTCTCTCTA |
| | TCGCTTCTGACCCCCGGTGCACCTGTGCCGAAACGCAAATGGGGAAACACCC |
| | GCTTTTCCCATGATTATGCATTGTCTCCACATTGTATGCTTCCAAGATTCTG |
| | GTGGGAATACTGCTGATAGCCTAACGTTCATGATCAAAATTTAACCCCTCTA |
| | ACCCCTACTTGACAGCAATATATAAACAGCCCGAAGCTGCCCTGTCTTAAAC |
| | CTTTTCCCTTATCATCATTATTAGCTTACTTTCATAATTGCGACTGGTTCCA |
| | ATTGACAAGCTTTTGATTTTAACGACTTTTAACTCTTACTAGATATATCAAA |
| | CTTCAAAGAATTCCGAAACG (SEQ ID NO: 25) |
| Synthetic | AACATCCAAAGACGAAAGGTTGAATGAAACCTTTTTGCCATCCGACATCCAC |
| MeOH- | AGGTCCATTCTCACACACCCGTGCCAAACGCAACAGGAGGGGATACACTAGC |
| inducible | AGCAGACCGTTGCAAACGCAGGACCTCCACTCCTCTTCTCCTCAACACCCAC |
| promoter | TTAGGCTACTAACACCATGACTTTATTAGCCTGTCTATCCTGGCCCCCCTGG |
| (4168092) | CGTTTTATTTCCGAATGCAACAAGCTCCGCATTACACCCGAACATCACTCCA |
| | GATGAGGGCTTTCTGAGTGTGGGGTCAAATAGTTTCATGTTCCCCAAATGGC |
| | CCAAAACTGACAGTTTAAACGCTGTCTTGGAACCTAATATGACAAAAGCGTG |
| | ATCTCATCCAAGATGAACTAAGTTTGGTTCGTTGAAATGCTAACGGCCAGTT |
| | GGTCAAAAAGAAACTTCCAAAAGTCGGCATACCGTTTGTCTTGTTTGGTATT |
| | GATTGACGAATGCTCAAAAATAATCTCATTAATGCTTAGCGCAGTCTCTCTA |
| | TCGCTTCTGAACCCCGGTGCACCTGTGCCGAAACGCAAATGGGGAACCCCCC |
| | GCTTTTTGGATGATTATGCATTGTCTCCACATTGTATGCTTCCAAGATTCTG |
| | GTGGGAATACTGCTGATAGCCTAACGTTCATGATCAAAATTTAACCCCTCTA |
| | ACCCCTACTTGACAGCAATATATAAACAGCCCGAAGCTGCCCTGTCTTAAAC |
| | CTTTTCCCTTATCATCATTATTAGCTTACTTTCATAATTGCGACTGGTTCCA |
| | ATTGACAAGCTTTTGATTTTAACGACTTTTAACTCTTACTAGATATATCAAA |
| | CTTCAAAGAATTCCGAAACG (SEQ ID NO: 26) |
| Synthetic | AACATCCAAAGACGAAAGGTTGAATGAAACCTTTTTGCCATCCGACATCCAC |
| MeOH- | AGGTCCATTCTCACACATAAGTGCCAAACGCAACAGGAGGGGATACACTAGC |
| inducible | AGCAGACCGTTGCAAACGCAGGACCTCCACTCCTCTTCTCCTCAACACCCAC |
| promoter | TTAGGCTACTAACACCATGACTTTATTAGCCTGTCTATCCTGGCCCCCCTGG |
| (4168094) | CGTTTTATTTCCGAATGCAACAAGCTCCGCATTACACCCGAACATCACTCCA |
| | GATGAGGGCTTTCTGAGTGTGGGGTCAAATAGTTTCATGTTCCCCAAATGGC |
| | CCAAAACTGACAGTTTAAACGCTGTCTTGGAACCTAATATGACAAAAGCGTG |
| | ATCTCATCCAAGATGAACTAAGTTTGGTTCGTTGAAATGCTAACGGCCAGTT |
| | GGTCAAAAAGAAACTTCCAAAAGTCGGCATACCGTTTGTCTTGTTTCCCATT |
| | GATTGACGAATGCTCAAAAATAATCTCATTAATGCTTAGCGCAGTCTCTCTA |
| | TCGCTTCTGACCCCCGGTGCACCTGTGCCGAAACGCAAATGGGGAAACACCC |
| | GCTTTTTGGATGATTATGCATTGTCTCCACATTGTATGCTTCCAAGATTCTG |
| | GTGGGAATACTGCTGATAGCCTAACGTTCATGATCAAAATTTAACCCCTCTA |
| | ACCCCTACTTGACAGCAATATATAAACAGCCCGAAGCTGCCCTGTCTTAAAC |
| | CTTTTCCCTTATCATCATTATTAGCTTACTTTCATAATTGCGACTGGTTCCA |
| | ATTGACAAGCTTTTGATTTTAACGACTTTTAACTCTTACTAGATATATCAAA |
| | CTTCAAAGAATTCCGAAACG (SEQ ID NO: 27) |
| Synthetic | AACATCCAAAGACGAAAGGTTGAATGAAACCTTTTTGCCATCCGACATCCAC |
| MeOH- | AGGTCCATTCTCACACATAAGTGCCAAACGCAACAGGAGGGGATACACTAGC |
| inducible | AGCAGACCGTTGCAAACGCAGGACCTCCACTCCTCTTCTCCTCAACACCCAC |
| promoter | TTAGGCTACTAACACCATGACTTTATTAGCCTGTCTATCCTGGCCCCCCTGG |
| (4168095) | CGTTTTATTTCCGAATGCAACAAGCTCCGCATTACACCCGAACATCACTCCA |
| | GATGAGGGCTTTCTGAGTGTGGGGTCAAATAGTTTCATGTTCCCCAAATGGC |
| | CCAAAACTGACAGTTTAAACGCTGTCTTGGAACCTAATATGACAAAAGCGTG |
| | ATCTCATCCAAGATGAACTAAGTTTGGTTCGTTGAAATGCTAACGGCCAGTT |
| | GGTCAAAAAGAAACTTCCAAAAGTCGGCATACCGTTTGTCTTGTTTGGTATT |
| | GATTGACGAATGCTCAAAAATAATCTCATTAATGCTTAGCGCAGTCTCTCTA |
| | TCGCTTCTGAACCCCGGTGCACCTGTGCCGAAACGCAAATGGGGAACCCCCC |
| | GCTTTTTGGATGATTATGCATTGTCTCCACATTGTATGCTTCCCCGATTCTG |
| | GTGGGAATACTGCTGATAGCCTAACGTTCATGATCAAAATTTAACCCCTCTA |
| | ACCCCTACTTGACAGCAATATATAAACAGCCCGAAGCTGCCCTGTCTTAAAC |
| | CTTTTCCCTTATCATCATTATTAGCTTACTTTCATAATTGCGACTGGTTCCA |
| | ATTGACAAGCTTTTGATTTTAACGACTTTTAACTCTTACTAGATATATCAAA |
| | CTTCAAAGAATTCCGAAACG (SEQ ID NO: 28) |
| Synthetic | AACATCCAAAGACGAAAGGTTGAATGAAACCTTTTTGCCATCCGACATCCAC |
| MeOH- | AGGTCCATTCTCACACATAAGTGCCAAACGCAACAGGAGGGGATACACTAGC |
| inducible | AGCAGACCGTTGCAAACGCAGGACCTCCACTCCTCTTCTCCTCAACACCCAC |
| promoter | TTAGGCTACTAACACCATGACTTTATTAGCCTGTCTATCCTGGCCCCCCTGG |
| (4168097) | CGTTTTATTTCCGAATGCAACAAGCTCCGCATTACACCCGAACATCACTCCA |
| | GATGAGGGCTTTCTGAGTGTGGGGTCAAATAGTTTCATGTTCCCCAAATGGC |
| | CCAAAACTGACAGTTTAAACGCTGTCTTGGAACCTAATATGACAAAAGCGTG |
| | ATCTCATCCAAGATGAACTAAGTTTGGTTCGTTGAAATGCTAACGGCCAGTT |
| | GGTCAAAAAGAAACTTCCAAAAGTCGGCATACCGTTTGTCTTGTTTGGTATT |
| | GATTGACGAATGCTCAAAAATAATCTCATTAATGCTTAGCGCAGCCCCTCTA |
| | TCGCTTCTGAACCCCGGTGCACCTGTGCCGAAACGCAAATGGGGAAACACCC |
| | GCTTTTTGGATGATTATGCATTGTCTCCACATTGTATGCTTCCAAGATTCTG |
| | GTGGGAATACTGCTGATAGCCTAACGTTCATGATCCCAATTTAACCCCTCTA |
| | ACCCCTACTTGACAGCAATATATAAACAGAAGGAAGCTGCCCCCCCTTAAAC |
| | CTTTTCCCTTATCATCATTATTAGCTTACTTTCATAATTGCGACTGGTTCCA |
| | ATTGACAAGCTTTTGATTTTAACGACTTTTAACTCTTACTAGATATATCAAA |
| | CTTCAAAGAATTCCGAAACG (SEQ ID NO: 29) |
| Synthetic | AACATCCAAAGACGAAAGGTTGAATGAAACCTTTTTGCCATCCGACATCCAC |
| MeOH- | AGGTCCATTCTCACACATAAGTGCCAAACGCAACAGGAGGGGATACACTAGC |
| inducible | AGCAGACCGTTGCAAACGCAGGACCTCCACTCCTCTTCTCCTCAACACCCAC |
| promoter | TTAGGCTACTAACACCATGACTTTATTAGCCTGTCTATCCTGGCCCCCCTGG |
| (4168098) | CGTTTTATTTCCGAATGCAACAAGCCCCGCATTACACCCGAACATCACTCCA |
| | GATGAGGGCTTTCTGAGTGTGGGGTCAAATAGTTTCATGTTCCCCAAATGGC |
| | CCAAAACTGACAGTTTAAACGCTGTCTTGGAACCTAATATGACAAAAGCGTG |
| | ATCTCATCCAAGATGAACTAAGTTTGGTTCGTTGAAATGCTAACGGCCAGTT |
| | GGTCAAAAAGAAACTTCCAAAAGTCGGCATACCGTTTGTCTTGTTTGGTATT |
| | GATTGACGAATGCTCAAAAATAATCTCATTAATGCTTAGCGCAGTCTCTCTA |
| | TCGCTTCTGAACCCCGGTGCACCTGTGCCGAAACGCAAATGGGGAAACACCC |
| | GCTTTTTGGATGATTATGCATTGTCTCCACATTGTATGCTTCCAAGATTCTG |
| | GTGGGAATACTGCTGATAGCCTAACGTTCATGATCAAAATTTAACCCCTCTA |
| | ACCCCTACTCCCCAGCAATATATAAACAGCCCGAAGCTGCCCTGTCTTAAAC |
| | CTTTTCCCTTATCATCATTATTAGCTTACTTTCATAATTGCGACTGGTTCCA |
| | ATTGACAAGCTTTTGATTTTAACGACTTTTAACTCTTACTAGATATATCAAA |
| | CTTCAAAGAATTCCGAAACG (SEQ ID NO: 30) |
| Synthetic | AACATCCAAAGACGAAAGGTTGAATGAAACCTTTTTGCCATCCGACATCCAC |
| MeOH- | AGGTCCATTCTCACACATAAGTGCCAAACGCAACAGGAGGGGATACACTAGC |
| inducible | AGCAGACCGTTGCAAACGCAGGACCTCCACTCCTCTTCTCCTCAACACCCAC |
| promoter | TTAGGCTACTAACACCATGACTTTATTAGCCTGTCTATCCTGGCCCCCCTGG |
| (4168101) | CGTTTTATTTCCGAATGCAACAAGCTCCGCATTACACCCGAACATCACTCCA |
| | GATGAGGGCTTTCTGAGTGTGGGGTCAAATAGTTTCATGTTCCCCAAATGGC |
| | CCAAAACTGACAGTTTAAACGCTGTCTTGGAACCTAATATGACAAAAGCGTG |
| | ATCTCATCCAAGATGAACTAAGTTTGGTTCGTTGAAATGCTAACGGCCCCCT |
| | GGTCAAAAAGAAACTTCCAAAAGTCGGCATACCGTTTGTCTTGTTTGGTATT |
| | GATTGACGAATGCTCAAAAATAATCTCATTAATGCTTAGCGCAGTCTCTCTA |
| | TCGCTTCTGACCCCCGGTGCACCTGTGCCGAAACGCAAATGGGGAAACACCC |
| | GCTTTTTGGATGATTATGCATTGTCTCCACATTGTATGCTTCCAAGATTCTG |
| | GTGGGAATACTGCTGATAGCCTAACGTTCATGATCAAAATTTAACCCCTCTA |
| | ACCCCTACTTGACAGCAATATATAAACAGCCCGAAGCTGCCCTGTCTTCCCC |
| | CTTTCCCTTTATCATCATTATTAGCTTACTTTCATAATTGCGACTGGTTCCA |
| | ATTGACAAGCTTTTGATTTTAACGACTTTTAACTCTTACTAGATATATCAAA |
| | CTTCAAAGAATTCCGAAACG (SEQ ID NO: 31) |
| Synthetic | AACATCCAAAGACGAAAGGTTGAATGAAACCTTTTTGCCATCCGACATCCAC |
| MeOH- | AGGTCCATTCTCACACATAAGTGCCAAACGCAACAGGAGGGGATACACTAGC |
| inducible | AGCAGACCGTTGCAAACGCAGGACCTCCACTCCTCTTCTCCTCAACACCCAC |
| promoter | TTAGGCTACTAACACCATGACTTTATTAGCCTGTCTATCCTGGCCCCCCTGG |
| (4168104) | CGTTTTATTTCCGAATGCAACAAGCTCCGCATTACACCCGAACATCACTCCA |
| | GATGAGGGCTTTCTGAGTGTGGGGTCAAATAGTTTCATGTTCCCCAAATGGC |
| | CCAAAACTGACAGTTTAAACGCTGTCTTGGAACCTAATATGACAAAAGCGTG |
| | ATCTCATCCAAGATGAACTAAGTTTGGTTCGTTGAAATGCTAACGGCCCCCT |
| | GGTCAAAAAGAAACTTCCAAAAGTCGGCATACCGTTTGTCTTGTTTGGTATT |
| | GATTGACGAATGCTCAAAAATAATCTCATTAATGCTTAGCGCAGTCTCTCTA |
| | TCGCTTCTGACCCCCGGTGCACCTGTGCCGAAACGCAAATGGGGAAACACCC |
| | GCTTTTTGGATGATTATGCATTGTCTCCACATTGTATGCTTCCAAGATTCTG |
| | GTGGGAATACTGCTGATAGCCTAACGTTCATGATCAAAATTTAACCCCTCTA |
| | ACCCCTACTTGACAGCAATATATAAACAGCCCGAAGCTGCCCTGTCTTAAAC |
| | CTTTTCCCTTATCATCATTATTAGCTTACTTTCATAATTGCGACTGGTTCCA |
| | ATTGACAAGCTTTTGATTTTAACGACTTTTAACTCTTACTAGATATATCAAA |
| | CTTCAAAGAATTCCGAAACG (SEQ ID NO: 32) |

Control promoter [P(AOX1)]

| | |
|---|---|
| P(AOX1) | AACATCCAAAGACGAAAGGTTGAATGAAACCTTTTTGCCATCCGACATCCACA |
| | GGTCCATTCTCACACATAAGTGCCAAACGCAACAGGAGGGGATACACTAGCAG |
| | CAGACCGTTGCAAACGCAGGACCTCCACTCCTCTTCTCCTCAACACCCACTTT |
| | TGCCATCGAAAAACCAGCCCAGTTATTGGGCTTGATTGGAGCTCGCTCATTCC |
| | AATTCCTTCTATTAGGCTACTAACACCATGACTTTATTAGCCTGTCTATCCTG |
| | GCCCCCCTGGCGAGGTTCATGTTTGTTTATTTCCGAATGCAACAAGCTCCGCA |
| | TTACACCCGAACATCACTCCAGATGAGGGCTTTCTGAGTGTGGGGTCAAATAG |
| | TTTCATGTTCCCCAAATGGCCCAAAACTGACAGTTTAAACGCTGTCTTGGAAC |
| | CTAATATGACAAAAGCGTGATCTCATCCAAGATGAACTAAGTTTGGTTCGTTG |
| | AAATGCTAACGGCCAGTTGGTCAAAAAGAAACTTCCAAAAGTCGGCATACCGT |
| | TTGTCTTGTTTGGTATTGATTGACGAATGCTCAAAAATAATCTCATTAATGCT |
| | TAGCGCAGTCTCTCTATCGCTTCTGAACCCCGGTGCACCTGTGCCGAAACGCA |
| | AATGGGGAAACACCCGCTTTTTGGATGATTATGCATTGTCTCCACATTGTATG |
| | CTTCCAAGATTCTGGTGGGAATACTGCTGATAGCCTAACGTTCATGATCAAAA |
| | TTTAACTGTTCTAACCCCTACTTGACAGCAATATATAAACAGAAGGAAGCTGC |
| | CCTGTCTTAAACCTTTTTTTTTATCATCATTATTAGCTTACTTTCATAATTGC |
| | GACTGGTTCCAATTGACAAGCTTTTGATTTTAACGACTTTTAACGACAACTTG |
| | AGAAGATCAAAAAACAACTAATTATTCGAAACG (SEQ ID NO: 33) |

### EQUIVALENTS

Those skilled in the art will recognize, or be able to ascertain using no more than routine experimentation, many equivalents to the specific embodiments of the invention described in this application. Such equivalents are intended to be encompassed by the following claims. The definitions provided in any one section of this application are intended to apply to any other section, where applicable.

We claim:

1. A synthetic promoter comprising a nucleic acid sequence as shown in SEQ ID NO: 1, wherein Y may be C or T, S may be G or C, and M may be A or C.

2. The synthetic promoter of claim 1, wherein (a) the nucleotide corresponding to position 32 is a C, or (b) the nucleotide corresponding to position 32 is a C, and all other Y bases in the sequence are T, all S bases in the sequence are G, and all M bases in the sequence are A.

3. The synthetic promoter of claim 1, wherein (a) the nucleotide corresponding to position 33 is a C, or (b) the nucleotide corresponding to position 33 is a C, and all other Y bases in the sequence are T, all S bases in the sequence are G, and all M bases in the sequence are A.

4. The synthetic promoter of claim 1, wherein (a) the nucleotide corresponding to position 70 is a C, or (b) the nucleotide corresponding to position 70 is a C, and all other Y bases in the sequence are T, all S bases in the sequence are G, and all M bases in the sequence are A.

5. The synthetic promoter of claim 1, wherein (a) the nucleotide corresponding to position 71 is a C, or (b) the nucleotide corresponding to position 71 is a C, and all Y bases in the sequence are T, all S bases in the sequence are G, and all other M bases in the sequence are A.

6. The synthetic promoter of claim 1, wherein (a) the nucleotide corresponding to position 72 is a C, or (b) the nucleotide corresponding to position 72 is a C, and all Y bases in the sequence are T, all S bases in the sequence are G, and all other M bases in the sequence are A.

7. The synthetic promoter of claim 1, wherein (a) the nucleotide corresponding to position 234 is a C, or (b) the nucleotide corresponding to position 234 is a C, and all Y bases in the sequence are T, all S bases in the sequence are G, and all other M bases in the sequence are A.

8. The synthetic promoter of claim 1, wherein (a) the nucleotide corresponding to position 413 is a C, or (b) the nucleotide corresponding to position 413 is a C, and all Y bases in the sequence are T, all S bases in the sequence are G, and all other M bases in the sequence are A.

9. The synthetic promoter of claim 1, wherein (a) the nucleotide corresponding to position 414 is a C, or (b) the nucleotide corresponding to position 414 is a C, and all Y bases in the sequence are T, all S bases in the sequence are G, and all other M bases in the sequence are A.

10. The synthetic promoter of claim 1, wherein (a) the nucleotide corresponding to position 415 is a C, or (b) the nucleotide corresponding to position 415 is a C, and all Y bases in the sequence are T, all S bases in the sequence are G, and all other M bases in the sequence are A.

11. The synthetic promoter of claim 1, wherein (a) the nucleotide corresponding to position 463 is a C, or (b) the nucleotide corresponding to position 463 is a C, and all Y bases in the sequence are T, all S bases in the sequence are G, and all other M bases in the sequence are A.

12. The synthetic promoter of claim 1, wherein (a) the nucleotide corresponding to position 464 is a C, or (b) the nucleotide corresponding to position 464 is a C, and all Y bases in the sequence are T, all S bases in the sequence are G, and all other M bases in the sequence are A.

13. The synthetic promoter of claim 1, wherein (a) the nucleotide corresponding to position 465 is a C, or (b) the nucleotide corresponding to position 465 is a C, and all Y bases in the sequence are T, all S bases in the sequence are G, and all other M bases in the sequence are A.

14. The synthetic promoter of claim 1, wherein (a) the nucleotide corresponding to position 513 is a C, or (b) the nucleotide corresponding to position 513 is a C, and all Y bases in the sequence are T, all S bases in the sequence are G, and all other M bases in the sequence are A.

15. The synthetic promoter of claim 1, wherein (a) the nucleotide corresponding to position 515 is a C, or (b) the nucleotide corresponding to position 515 is a C, and all Y bases in the sequence are T, all S bases in the sequence are G, and all other M bases in the sequence are A.

16. The synthetic promoter of claim 1, wherein (a) the nucleotide corresponding to position 531 is a C, or (b) the nucleotide corresponding to position 531 is a C, and all Y bases in the sequence are T, all S bases in the sequence are G, and all other M bases in the sequence are A.

17. The synthetic promoter of claim 1, wherein (a) the nucleotide corresponding to position 567 is a C, or (b) the nucleotide corresponding to position 567 is a C, and all Y bases in the sequence are T, all S bases in the sequence are G, and all other M bases in the sequence are A.

18. The synthetic promoter of claim 1, wherein (a) the nucleotide corresponding to position 569 is a C, or (b) the nucleotide corresponding to position 569 is a C, and all Y bases in the sequence are T, all S bases in the sequence are G, and all other M bases in the sequence are A.

19. The synthetic promoter of claim 1, wherein (a) the nucleotide corresponding to position 579 is a C, or (b) the nucleotide corresponding to position 579 is a C, and all Y bases in the sequence are T, all S bases in the sequence are G, and all other M bases in the sequence are A.

20. The synthetic promoter of claim 1, wherein (a) the nucleotide corresponding to position 580 is a C, or (b) the nucleotide corresponding to position 580 is a C, and all Y bases in the sequence are T, all S bases in the sequence are G, and all other M bases in the sequence are A.

21. The synthetic promoter of claim 1, wherein (a) the nucleotide corresponding to position 581 is a C, or (b) the nucleotide corresponding to position 581 is a C, and all Y bases in the sequence are T, all S bases in the sequence are G, and all other M bases in the sequence are A.

22. The synthetic promoter of claim 1, wherein (a) the nucleotide corresponding to position 616 is a C, or (b) the nucleotide corresponding to position 616 is a C, and all Y bases in the sequence are T, all S bases in the sequence are G, and all other M bases in the sequence are A.

23. The synthetic promoter of claim 1, wherein (a) the nucleotide corresponding to position 617 is a C, or (b) the nucleotide corresponding to position 617 is a C, and all Y bases in the sequence are T, all S bases in the sequence are G, and all other M bases in the sequence are A.

24. The synthetic promoter of claim 1, wherein (a) the nucleotide corresponding to position 660 is a C, or (b) the nucleotide corresponding to position 660 is a C, and all Y bases in the sequence are T, all S bases in the sequence are G, and all other M bases in the sequence are A.

25. The synthetic promoter of claim 1, wherein (a) the nucleotide corresponding to position 661 is a C, or (b) the nucleotide corresponding to position 661 is a C, and all Y bases in the sequence are T, all S bases in the sequence are G, and all other M bases in the sequence are A.

26. The synthetic promoter of claim 1, wherein (a) the nucleotide corresponding to position 686 is a C, or (b) the nucleotide corresponding to position 686 is a C, and all Y bases in the sequence are T, all S bases in the sequence are G, and all other M bases in the sequence are A.

27. The synthetic promoter of claim 1, wherein (a) the nucleotide corresponding to position 687 is a C, or (b) the nucleotide corresponding to position 687 is a C, and all Y bases in the sequence are T, all S bases in the sequence are G, and all other M bases in the sequence are A.

28. The synthetic promoter of claim 1, wherein (a) the nucleotide corresponding to position 688 is a C, or (b) the nucleotide corresponding to position 688 is a C, and all Y bases in the sequence are T, all S bases in the sequence are G, and all other M bases in the sequence are A.

29. The synthetic promoter of claim 1, wherein (a) the nucleotide corresponding to position 706 is a C, or (b) the nucleotide corresponding to position 706 is a C, and all Y bases in the sequence are T, all S bases in the sequence are G, and all other M bases in the sequence are A.

30. The synthetic promoter of claim 1, wherein (a) the nucleotide corresponding to position 707 is a C, or (b) the nucleotide corresponding to position 707 is a C, and all Y bases in the sequence are T, all S bases in the sequence are G, and all other M bases in the sequence are A.

31. The synthetic promoter of claim 1, wherein (a) the nucleotide corresponding to position 708 is a C, or (b) the nucleotide corresponding to position 708 is a C, and all Y bases in the sequence are T, all S bases in the sequence are G, and all other M bases in the sequence are A.

32. The synthetic promoter of claim 1, wherein (a) the nucleotide corresponding to position 719 is a C, or (b) the nucleotide corresponding to position 719 is a C, and all Y bases in the sequence are T, all S bases in the sequence are G, and all other M bases in the sequence are A.

33. The synthetic promoter of claim 1, wherein (a) the nucleotide corresponding to position 720 is a C, or (b) the nucleotide corresponding to position 720 is a C, and all Y bases in the sequence are T, all S bases in the sequence are G, and all other M bases in the sequence are A.

34. The synthetic promoter of claim 1, wherein (a) the nucleotide corresponding to position 721 is a C, or (b) the nucleotide corresponding to position 721 is a C, and all Y bases in the sequence are T, all S bases in the sequence are G, and all other M bases in the sequence are A.

35. The synthetic promoter of claim 1, wherein (a) the nucleotide corresponding to position 725 is a C, or (b) the nucleotide corresponding to position 725 is a C, and all Y bases in the sequence are T, all S bases in the sequence are G, and all other M bases in the sequence are A.

36. The synthetic promoter of claim 1, wherein (a) the nucleotide corresponding to position 726 is a C, or (b) the nucleotide corresponding to position 726 is a C, and all Y bases in the sequence are T, all S bases in the sequence are G, and all other M bases in the sequence are A.

37. The synthetic promoter of claim 1, wherein (a) the nucleotide corresponding to position 727 is a C, or (b) the nucleotide corresponding to position 727 is a C, and all Y bases in the sequence are T, all S bases in the sequence are G, and all other M bases in the sequence are A.

38. The synthetic promoter of claim 1, wherein (a) the nucleotide corresponding to position 733 is a C, or (b) the nucleotide corresponding to position 733 is a C, and all Y bases in the sequence are T, all S bases in the sequence are G, and all other M bases in the sequence are A.

39. The synthetic promoter of claim 1, wherein (a) the nucleotide corresponding to position 736 is a C, or (b) the nucleotide corresponding to position 736 is a C, and all Y bases in the sequence are T, all S bases in the sequence are G, and all other M bases in the sequence are A.

40. A synthetic promoter comprising a polynucleotide having one to thirty-eight bases different than SEQ ID NO: 33, wherein the one to thirty-eight bases that are different are located at position(s) 32, 33, 70, 71, 72, 313, 492, 493, 494, 542, 543, 544, 592, 594, 610, 646, 648, 658, 659, 660, 695, 696, 739, 740, 765, 766, 767, 785, 786, 787, 798, 799, 800, 804, 805, 806, 812, and/or 815 of a nucleic sequence as shown in SEQ ID NO: 33.

41. A synthetic promoter comprising a polynucleotide having at least 90%, at least 95%, or at least 99% identity to a nucleic sequence as shown in any one of SEQ ID NOs: 2-32.

42. A synthetic promoter comprising a polynucleotide having no more than 38 substitutions relative to a nucleic sequence as shown in any one of SEQ ID NOs: 2-32.

43. A synthetic promoter having a nucleic sequence as shown in any one of SEQ ID NOs: 2-32.

44. A transcriptional unit comprising the synthetic promoter according to any one of claims 1-43.

45. The transcriptional unit of claim 44, wherein the synthetic promoter is operably linked to one or more genes of interest.

46. The transcriptional unit of claim 45, wherein the synthetic promoter is operably linked to 1, 2, 3, 4, 5, 6, 7, 8, 9, or 10 genes of interest.

47. The transcriptional unit of claim 45, wherein the synthetic promoter is operably linked to one gene of interest.

48. The transcriptional unit of claim 45, wherein the synthetic promoter is operably linked to four genes of interest.

49. The transcriptional unit of claim 45, wherein the synthetic promoter is operably linked to eight genes of interest.

50. The transcriptional unit of any one of claims 45-49, wherein the gene of interest is expressed as an RNA.

51. The transcriptional unit of any one of claims 45-49, wherein the gene of interest encodes a protein.

52. The transcriptional unit of claim 51, wherein the protein is an enzyme.

53. The transcriptional unit of claim 51, wherein the protein is vaccinia capping enzyme, T7 polymerase, or O-methyltransferase.

54. The transcriptional unit of any one of claims 45-49, wherein the gene of interest encodes Dp1B silk protein, gelatin mouse α1(I), gelatin mouse α(III), collagen human type III, cellulase, alpha-amylase, E. coli phytase, T. aquaticus subtilisin, human serum albumin, human insulin, bovine β-caesin, pertussis pertactin, tetani tetanus toxin fragment C, strep T7A endoglucanase, Aspergillus catalase L, Sc invertase, tumor necrosis factor (TNF) alpha, bovine a-lactalbumin, or bovine α-lactalbumin.

55. A host cell comprising one or more synthetic promoters according to any one of claims 1-43 and/or one or more transcriptional units according to any one of claims 44-54.

56. The host cell of claim 55, wherein the host cell is methylotrophic.

57. The host cell of claim 55 or claim 56, wherein the host cell is a yeast cell.

58. The host cell of claim 57, wherein the host cell is from a genus of: Pichia, Komagataella, Hansenula, or Candida.

59. The host cell of claim 58, wherein the host cell is Pichia pastoris, Pichia pseudopastoris, Komagataella phaffii, Pichia stipitis, Pichia membranifaciens, Komagataella pseudopastoris, Komagataella pastoris, Komagataella kurtzmanii, Komagataella mondaviorum, Hansenula polymorpha, Candida boidinii, or Pichia methanolica.

60. The host cell of claim 59, wherein the host cell is Pichia pastoris.

61. The host cell of any one of claims 55-60, wherein one or more synthetic promoters according to any one of claims 1-43 and/or one or more transcriptional units according to any one of claims 44-54 are integrated into the genome of the host cell.

62. A method of engineering a host cell for protein expression comprising transforming the host cell with one or more synthetic promoters according to any one of claims 1-43 and/or one or more transcriptional units according to any one of claims 44-54.

63. A method of expressing a gene of interest or producing a molecule of interest, the method comprising culturing a host cell comprising one or more synthetic promoters according to any one of claims 1-43 and/or one or more transcriptional units according to any one of claims 44-54 in a suitable medium.

64. The method of claim 63, wherein the one or more synthetic promoters according to any one of claims 1-43 and/or one or more transcriptional units according to any one of claims 44-54 are integrated into the genome of the host cell.

65. The method of claim 63 or claim 64, wherein the gene of interest encodes a protein.

66. The method of claim 65, wherein the protein is an enzyme.

67. The method of claim 65, wherein the protein is vaccinia capping enzyme, T7 polymerase, or O-methyltransferase.

68. The method of claim 63 or claim 64, wherein the gene of interest encodes dp1B silk protein, gelatin mouse α1(I), gelatin mouse α(III), collagen human type III, cellulase, alpha-amylase, E. coli phytase, T. aquaticus subtilisin, human serum albumin, human insulin, bovine β-caesin, pertussis pertactin, tetani tetanus toxin fragment C, strep T7A endoglucanase, Aspergillus catalase L, Sc invertase, tumor necrosis factor (TNF) alpha, bovine a-lactalbumin, or bovine α-lactalbumin.

69. The method of any one of claims 63-68, further comprising extracting the expressed protein, RNA, or molecule of interest from biomass.

70. The method of any one of claims 63-68, further comprising collecting the expressed protein, RNA, or molecule of interest from culture, culture medium, cell-free spent culture medium, and/or cell-containing culture medium.

71. The method of any one of claims 63-70, wherein the one or more synthetic promoters are methanol-inducible.
